(19) United States
(12) Patent Application Publication
(10) Pub. No.: US 2023/0137723 A1
(43) Pub. Date: May 4, 2023

(54) MESENCHYMAL STEM CELLS FOR THE PREVENTION AND TARGETED TREATMENT OF CANCER AND OTHER DISORDERS

(71) Applicant: MAM Holdings of West Florida, L.L.C., Palm Harbor, FL (US)

(72) Inventor: Carl Randall Harrell, Tarpon Springs, FL (US)

(21) Appl. No.: 17/977,476

(22) Filed: Oct. 31, 2022

Related U.S. Application Data
(60) Provisional application No. 63/274,382, filed on Nov. 1, 2021.

(51) Int. Cl. A61K 35/28; A61P 35/00; A61K 45/06; A61P 37/02

(52) U.S. Cl. CPC A61K 35/28; A61P 35/00; A61K 45/06; A61P 37/02

(57) ABSTRACT

Disclosed herein are compositions, formulations, and/or methods of using mesenchymal stem cells (MSCs) for preventing and treating cancer, and for suppressing the growth or proliferation of cancer. The MSCs contain significant numbers of anti-tumor compounds, including, for instance, growth factors, anti-inflammatory cytokines, and the like, and are amenable for long-term storage without the loss of biological potency. In at least one embodiment, various types of MSCs are shown to improve survival of tumor bearing animals. In at least another embodiment, one or more types of MSCs are used in combination with, or formulated with, one or more additional active agents.

### CROSS-REFERENCE TO RELATED APPLICATION

This application claims priority to U.S. Provisional Application No. 63/274,382, filed Nov. 1, 2021, which is hereby incorporated by reference in its entirety.

### FIELD OF THE DISCLOSURE

The disclosure relates generally to compositions, formulations, and methods for immunotherapy. In particular, embodiments of the disclosure relate to one or more types of mesenchymal stem cells as a targeted therapy for the prevention and treatment of cancers, tumors, and various associated disorders. One or more compositions and/or formulations described herein may be used in combination with, or formulated with, one or more additional active agents.

### BACKGROUND

During the last three decades, immunosuppressive drugs have been frequently used in clinical practice due to the increase of autoimmune and inflammatory diseases. However, long-term use of immunosuppressive agents may result in the development of severe infections due to the inhibition of anti-microbial immune response. As a result, one area of interest, especially in the field of cancer immunotherapy, is the development of novel immunomodulatory compounds that inhibit detrimental immune responses without causing life-threatening immunosuppression.

Mesenchymal stem cells (“MSC” or “MSCs”) are self-renewable, multipotent stem cells that regulate innate and/or adaptive immune responses in various human tissues. For instance, MSCs play a role in responding to tissue injury and reducing inflammation. Moreover, due to their immunosuppressive properties, MSCs have therapeutic potential in alleviating various diseases (e.g., autoimmune diseases, specific cancers).

MSCs may originate from different sources (e.g., bone marrow, amniotic fluid, placental tissue, etc.) and contain a variety of biological compounds (e.g., carbohydrates, proteins and peptides, lipids, lactate, pyruvate, electrolytes, enzymes, hormones, and various growth factors).

MSCs are also constituents of the cellular environment existing around various tumors. Thus, in the specific context of various cancers, MSCs may have the potential to modulate the phenotype and/or function of one or more types of immune cells that participate in anti-tumor immune responses.

Given the foregoing, there exists a significant need for systems and methods that treat one or more diseases using MSCs and/or MSC-derived compounds. In particular, there is a need for methods that provide for the clinical use of MSCs in cancer immunotherapy.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

Embodiments of the present disclosure are directed towards compositions, formulations, and methods for using one or more types of mesenchymal stem cells (MSCs) for preventing and treating cancer, and for suppressing the growth or proliferation of cancer. The MSCs contain significant numbers of anti-tumor compounds, including, for instance, growth factors, anti-inflammatory cytokines, and the like, and are amenable for long-term storage without the loss of biological potency. In at least one embodiment, various types of MSCs are shown to improve survival of tumor bearing animals. In at least another embodiment, one or more types of MSCs are used in combination with, or formulated with, one or more additional active agents.

In at least a further embodiment, the aforementioned one or more types of MSCs suppress the production of inflammatory cytokines and promote the secretion of immunosuppressive immune responses and/or immune cell phenotypes. In at least another embodiment, the one or more types of MSCs favor the development of tolerogenic and/or regulatory phenotypes in activated monocytes and lymphocytes, indicating its potential for therapeutic use in the alleviation of various cancers.

In at least another embodiment, the aforementioned one or more types of MSCs contain anti-tumor compounds (e.g., various cytokines) that enhance one or more immune responses (e.g., T-cell driven responses) in a tumor microenvironment. In at least another embodiment, a method for prevention and treatment of cancers is disclosed, which includes, for instance, altering the response of endogenous immune cells in the subject provided. The method may therefore comprise administering to a subject an effective amount of one or more types of MSCs, which may be composed within one or more MSC compositions and/or formulations, thereby altering the response of one or more endogenous immune cells (e.g., dendritic cells, macrophages, natural killer cells, T cells, and the like) in the subject. In at least another embodiment, embodiments, administration of an effective amount of such one or more types of MSCs increases the likelihood of survival of the subject and/or decreases the incidence of cancers and/or tumors in the subject. Further, administering the one or more types of MSCs can reduce tumor weight and/or tumor volume in a subject with cancer.

In at least another embodiment, the one or more types of MSCs may be administered in combination with one or more agents, such as, for instance, one or more antimicrobial agents, one or more analgesic agents, one or more local anesthetic agents, one or more anti-inflammatory agents, one or more anti-oxidant agents, one or more immunosuppressant agents, one or more anti-allergenic agents, one or more enzyme cofactors, one or more essential nutrients, one or more growth factors, and combinations thereof.

In at least another embodiment, the one or more types of MSCs are used as a delivery vehicle for one or more other agents, including, for instance, bi-specific T-cell engaging antibodies, glypican 3, one or more treatment compounds (e.g., prodrugs, anti-cancer drugs, including, for instance, one or more chemotherapeutic drugs, and the like), one or more cytokines (e.g., IL-2, IL-12, IL-21, and TRAIL), one or more interferons (e.g., IFN-α, IFN-β, and IFN-γ), and combinations thereof.

In at least another embodiment, a pharmaceutical composition comprises one or more types of MSCs and one or more pharmaceutically acceptable excipients. Such a composition may comprise one or more agents selected from the group consisting of adjuvants, antioxidants, anti-inflammatory agents, growth factors, neuroprotective agents, antimicrobial agents, local anesthetics, and combinations thereof. In at least another embodiment, the composition may comprise exosomes generated ex vivo from MSCs. Such exosomes may be used as a delivery vehicle for one or more MSC-sourced biological molecules (e.g., anti-tumorigenic miRNAs, messenger RNAs (mRNAs), enzymes, cytokines, chemokines, growth factors, immunomodulatory factors, small-molecule drugs, proteins, and combinations thereof. These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows various mechanisms of MSC-mediated suppression of anti-tumor immunity.

FIG. 2 shows various MSC-based therapies for treating cancers and/or tumors, including administration of one or more types of MSCs and/or administration of one or more types of MSC-derived exosomes, to alter one or more responses of one or more immune cells in a subject, according to at least one embodiment of the disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may affect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a,” “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise,” “comprises,” and “comprising” are to be interpreted inclusively rather than exclusively. Likewise, the terms “include,” “including,” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of.” Although having distinct meanings, the terms “comprising,” “having,” “containing,” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

### Definitions

The following is a non-exhaustive and non-limiting list of terms used herein and their respective definitions.

The terms “agent” or “active agent,” which are used interchangeably herein, refer to a physiologically or pharmacologically active substance that acts locally and/or systemically in a subject's body. An “agent” or “active agent” is a compound or substance that is administered to an individual for the treatment (e.g., therapeutic agent, cancer therapeutic agent, and the like), prevention (e.g., prophylactic agent), or diagnosis (e.g., diagnostic agent) of a disease or disorder. Such agents may also include therapeutics that prevent or alleviate symptoms, such as, for instance, symptoms associated with one or more cancers or treatments for such cancer(s).

The term “administering” or “administration” refers to providing or giving a subject one or more agents and/or formulations, such as one or more types of MSCs, either alone or in conjunction with any other compound and/or agent (including, e.g., cancer prophylactic or anti-cancer therapeutic agents), by any effective route. Exemplary routes of administration include, but are not limited to, injection (such as, e.g., subcutaneous, subdermal, intramuscular, intradermal, intraperitoneal, intracerebroventricular, intraosseous, intratumoral, intraprostatic, and intravenous), transdermal, intranasal, oral, vaginal, rectal, and inhalation.

The term “amniotic factor” generally refers to one or more compounds naturally present in the amniotic fluid. These include, for example, carbohydrates, proteins and peptides (e.g., enzymes, hormones), lipids, metabolic substrates and products (e.g., lactate, pyruvate), and electrolytes.

The term “antigen” refers to a compound, composition, and/or substance that can stimulate the production of antibodies or an immune response in a subject, including compositions (such as one that includes a tumor-specific protein) that are injected or absorbed into a subject. An “antigen” may react with the products of specific humoral and/or cellular immunity, including, for example, those induced by heterologous antigens.

The term “cancer” refers to a class of diseases or conditions in which abnormal cells divide without control and can invade nearby tissues. A malignant cancer is one in which a group of tumor cells display one or more of uncontrolled growth (e.g., division beyond normal limits), invasion (e.g., intrusion on and destruction of adjacent tissues), and/or metastasis (e.g., spread to other locations in the subject's body via lymph or blood). As used herein, the terms “metastasis” or “metastasize” refer to the spread of cancer from one part of the body to another. A tumor formed by cells that have spread is called a “metastatic tumor” or a “metastasis.” The metastatic tumor contains cells that are similar to those in the original tumor (i.e., the tumor at the primary site of tumor growth). A “cancer cell” or “tumor cell” refers to an individual cell of a cancerous growth or tissue. A “tumor” refers generally to a swelling or lesion formed by an abnormal growth of cells, which may be benign, pre-malignant, or malignant. Most cancers form tumors, but some, e.g., leukemia, and some blood cancers, do not necessarily form tumors. For those cancers that form tumors, the terms “cancer,” “cancer cell,” “tumor,” and “tumor cell” are used interchangeably. The amount of a tumor in a given subject is the “tumor burden,” which can be measured as the number, volume, and/or weight of the tumor.

Exemplary tumors, such as cancers, that can be treated using the disclosed one or more agents, formulations, and/or methods (e.g., including one or more types of MSCs, either alone or in conjunction with one or more other agents) include solid tumors, such as breast carcinomas (e.g., lobular and duct carcinomas, such as a triple negative breast cancer), sarcomas, carcinomas of the lung (e.g., non-small cell carcinoma, large cell carcinoma, blood cancers, squamous carcinoma, and adenocarcinoma), mesothelioma of the lung, colorectal adenocarcinoma, stomach carcinoma, prostatic adenocarcinoma, ovarian carcinoma (e.g., serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (e.g., transitional cell carcinoma, adenocarcinoma, and squamous carcinoma), renal cell adenocarcinoma, endometrial carcinomas (e.g., adenocarcinomas and mixed Mullerian tumors (carcinosarcomas)), carcinomas of the endocervix, ectocervix, and vagina (e.g., adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors, and various types of sarcomas and Merkel cell carcinoma), esophageal carcinoma, carcinomas of the nasopharynx and oropharynx (e.g., squamous carcinoma and adenocarcinomas of same), salivary gland carcinomas, brain and central nervous system tumors (e.g., tumors of glial, neuronal, and meningeal origin), tumors of peripheral nerve, soft tissue sarcomas and sarcomas of bone and cartilage, head and neck squamous cell carcinoma, and lymphatic tumors (e.g., B-cell and T-cell malignant lymphoma). In one example, the tumor is an adenocarcinoma. In another example, the cancer is pancreatic adenocarcinoma. In yet another example, the cancer is colorectal adenocarcinoma. The disclosed methods and/or formulations can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CIVIL), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

The term “combination therapy” refers to the administration of different compounds, agents, and/or individual therapies in a sequential and/or simultaneous manner. Individual elements of a “combination therapy” may be administered at different times and/or by different routes, but act in combination to provide a beneficial effect on the subject.

The term “compound” refers to a substance formed from one or more chemical elements, arranged together in any proportion or structural arrangement. The one or more chemical elements may be either naturally occurring and/or non-naturally occurring. As used herein, the term “biological compound” refers to a compound of biological origin and/or having one or more effects on a subject's local and/or systemic biological functions. Accordingly, “compounds” or “biological compounds” include, as non-limiting examples, various proteins (e.g., growth factors, hormones, enzymes), nucleic acids, and pharmaceutical products (e.g., drugs, prodrugs). The term “drug” generally refers to a medicine or other substance that has a physiological effect when introduced into a subject. The term “prodrug” generally refers to a biologically and/or chemically inactive compound that can be metabolized by a subject to produce a drug.

The terms “decrease,” “lower,” “lessen,” “reduce,” and “abate,” which are used interchangeably herein, refer generally to the ability of a compound, formulation, or therapy (including those disclosed herein) to produce, elicit, and/or cause a lesser physiological response (e.g., downstream effects) compared to the response caused by a respective control compound, formulation, or therapy. A “decrease” or “reduced” amount is typically a “statistically significant” amount, and may include a decrease that is, for instance, 1.1, 1.2, 1.5, 2, 3, 4, 5, 6, 7, 8, 9, 10, 15, 20, or 30 or more times (e.g., 500, 1000 times) (including all integers and decimal points in between and above 1, e.g., 1.5, 1.6, 1.7, 1.8, etc.).

The term “dendritic cell” refers to a type of specialized antigen-presenting cell (“APC”) involved in innate and/or adaptive immunity. Dendritic cells may also be referred to herein as “DC” or “DCs.” Dendritic cells may be present in the tumor microenvironment, and these are referred to as “tumor-associated dendritic cells” (“tDC” or “tDCs”).

The terms “effective amount” or “therapeutically effective amount,” which are used interchangeably herein, refer to the amount of an agent (e.g., including one or more types of MSCs described herein) that is sufficient to effect beneficial or desired therapeutic result, including clinical results. An “effective amount” may vary depending upon one or more of: the subject and disease condition being treated, the sex, weight and age of the subject, the severity of the disease condition, the manner of administration, the ability of one or more formulations to elicit a desired response in the subject, and the like. The beneficial therapeutic effect can include, but is not limited to, enablement of diagnostic determinations; prevention of disease or tumor formation; amelioration of a disease, symptom, disorder, and/or pathological condition; reducing or preventing the onset of a disease, symptom, disorder, and/or pathological condition; and generally counteracting a disease, symptom, disorder, and/or pathological condition. The term “effective amount” includes an amount that is effective to “treat” a subject (e.g., a patient or individual). When a therapeutic amount is indicated, the precise amount of one or more formulations described in the present disclosure to be administered can be determined by a physician, based on, for instance, considerations such as individual differences in age, weight, tumor size, extent of infection or metastasis, and/or condition of the subject (individual).

In at least one embodiment, an “effective amount” (e.g., of one or more agents and/or formulations described herein, including one or more types of MSCs, either alone or in conjunction with one or more other agents) may be an amount sufficient to increase the rate of survival of a subject, reduce the volume/size of a tumor, reduce the weight of a tumor, reduce the number/extent of metastases, reduce the volume/size of a metastasis, reduce the weight of a metastasis, and combinations thereof, for example by at least about 10%, at least about 20%, at least about 25%, at least about 50%, at least about 70%, at least about 75%, at least about 80%, at least about 90%, at least about 95%, or at least about 99% (as compared to no administration of the therapeutic agent and/or formulation). In at least a further embodiment, an “effective amount” (e.g., of one or more agents and/or formulations described herein, including one or more types of MSCs, either alone or in conjunction with one or more other agents) may be an amount sufficient to increase the survival time of a subject, such as a subject with cancer, for example by at least about 10%, at least about 20%, at least about 25%, at least about 50%, at least about 70%, at least about 75%, at least about 80%, at least about 90%, at least about 95%, 100%, 200%, 300%, 400%, or 500% (as compared to no administration of the therapeutic agent and/or formulation).

The terms “enhance,” “induce,” “induction,” and “increase,” which are used interchangeably herein, refer generally to the ability of a compound, formulation, or therapy (including those disclosed herein) to produce, elicit, and/or cause a greater physiological response (e.g., downstream effects) compared to the response caused by a respective control compound, formulation, or therapy. A non-limiting example of a measurable physiological response includes inducing one or more responses of cancer-associated endogenous immune cells in the subject and/or an increase in cytotoxic and/or cancer cell death killing ability, among others apparent from the description herein. An “enhanced” or “increased” amount is typically a “statistically significant” amount, and may include an increase that is, for instance, 1.1, 1.2, 1.5, 2, 3, 4, 5, 6, 7, 8, 9, 10, 15, 20, or 30 or more times (e.g., 500, 1000 times) (including all integers and decimal points in between and above 1, e.g., 1.5, 1.6, 1.7, 1.8, etc.).

The term “growth factor” refers to any compound (e.g., one or more groups of proteins or hormones) that stimulate cellular growth. Generally, growth factors play an important role in promoting cellular differentiation and cell division, and they occur in a wide range of organisms, including humans.

The term “immune cell” refers to any cell of the immune system that has one or more effector functions (e.g., cytotoxic cell killing activity, secretion of cytokines, induction of antibody-dependent cell-mediated cytotoxicity (ADCC), and/or induction of complement-dependent cytotoxicity (CDC)).

The terms “immunologic,” “immunological,” or “immune” response, which are used interchangeably herein, refer to the development of a beneficial humoral (i.e., antibody-mediated) and/or a cellular (e.g., mediated by immune cells, such as antigen-specific T cells, or their secretion products) response directed against an antigen and/or immunogen in a specific subject. Such a response can be an active response induced by administration of an antigen and/or immunogen, or a passive response induced by administration of antibodies or primed T-cells. A cellular immune response is elicited by the presentation of polypeptide epitopes in association with Class I or Class II major histocompatibility complex (MHC) molecules to activate antigen-specific CD4+ healer T cells and/or cos+ cytotoxic T cells. The response may also involve, for instance, activation of monocytes, macrophages, natural killer (NK) cells, basophils, dendritic cells, astrocytes, microglia cells, eosinophils, and/or other components of innate immunity. The presence of a cell-mediated immunological response can be determined by proliferation assays (e.g., CD4+ T cells) or cytotoxic T lymphocyte (CTL) assays. The relative contributions of humoral and cellular responses to the protective or therapeutic effect of an antigen and/or immunogen can be distinguished by, for example, separately isolating antibodies and T cells from an immunized syngeneic animal and measuring the protective or therapeutic effect in a second subject.

The term “ionizing radiation” refers to radiation, traveling as a particle or electromagnetic wave, that carries sufficient energy to detach electrons from atoms or molecules, thereby ionizing an atom or a molecule. Generally, ionizing radiation is made up of energetic subatomic particles, ions, or atoms moving at high speeds and electromagnetic waves on the high-energy end of the electromagnetic spectrum. Radiation has been demonstrated to induce adaptive immune responses to mediate tumor regression. In addition, the induction of type I interferons (“IFNs”) by radiation is essential for the function of CD8+ T cells. Radiation induces cell stress and causes excess deoxyribonucleic acid (DNA) breaks, indicating that the nucleic acid-sensing pathway likely accounts for the induction of type I IFNs upon radiation. Type I IFN responses in DCs dictate the efficacy of antitumor radiation. In contrast, chemotherapeutic agents and anti-human epidermal growth factor receptor 2 (HER2) antibody treatments have been demonstrated to depend on a distinct immune mechanism to trigger adaptive immune responses. In general, therapeutic radiation-mediated antitumor immunity depends on a proper cytosolic DNA sensing pathway. In at least one embodiment, one or more agents, formulations, and/or methods (e.g., including one or more types of MSCs, either alone or in conjunction with one or more other agents) described herein is administered in combination with radiation therapy.

The term “macrophage” refers to a type of white blood cell of the immune system that engulfs and digests cellular debris, foreign substances, microbes, cancer cells, and the like. These phagocytes include various subtypes (e.g., histiocytes, Kupffer cells, alveolar macrophages, microglia, and others), but all are part of the mononuclear phagocyte system. Besides phagocytosis, macrophages play a critical role in both innate and adaptive immunity by recruiting other endogenous immune cells (e.g., lymphocytes). For example, they are important as antigen presenters to T cells. In humans, dysfunctional macrophages can cause severe diseases (e.g., chronic granulomatous disease) that result in frequent infections. Beyond increasing inflammation and stimulating the immune system, macrophages also play an important anti-inflammatory role and can decrease immune reactions through the release of various compounds (e.g., cytokines). Macrophages that encourage inflammation may be termed “M1 macrophages” because they have the so-called “M1 phenotype,” whereas those that decrease inflammation and encourage tissue repair may be termed “M2 macrophages” because they have the so-called “M2 phenotype.”

The term “parenteral administration” refers to a type of administration by any method other than through the digestive tract or non-invasive topical or regional routes. As a non-limiting example, parenteral administration may include administration to a subject via intravenous, intradermal, intraperitoneal, intrapleural, intratracheal, intraarticular, intrathecal, intramuscular, subcutaneous, subjunctival, injection, and/or infusion.

The term “peptide” refers to a polymer of amino acid residues. The amino acid residues may be naturally occurring and/or non-naturally occurring. The terms “polypeptide,” “peptide,” and “protein” are used interchangeably herein. The terms apply to, for instance, amino acid polymers of one or more amino acid residues, an artificial chemical mimetic of a corresponding naturally occurring amino acid, naturally occurring amino acid polymers, and non-naturally occurring amino acid polymers.

The terms “subject,” “individual,” or “patient,” which are used interchangeably herein, refer to a vertebrate, such as a mammal (e.g., a human). Mammals include, but are not limited to, murines (e.g., mice), simians, humans, farm animals, sport animals, and pets. In at least one embodiment, the subject is a non-human mammal, such as a monkey or other non-human primate, mouse, rat, rabbit, guinea pig, pig, goat, sheep, dog, cat, horse, or cow. In at least one example, the subject has a tumor, such as a cancer, that can be treated using one or more agents, formulations, and/or methods (e.g., including one or more types of MSCs, either alone or in conjunction with one or more other agents) disclosed herein. In at least an additional example, the subject is a laboratory animal/organism, such as, for example, a mouse, rabbit, guinea pig, or rat. In at least a further example, a subject includes, for instance, farm animals, domestic animals and/or pets (e.g., cats, dogs). In at least a still further example, a subject is a human patient that has a cancer, has been diagnosed with a cancer, and/or is at risk of having a cancer. A “patient” can specifically refer to a subject that has been diagnosed with a particular disease, condition, and/or indication that can be treated with refers to a subject that has been diagnosed with a particular indication that can be treated with one or more agents, formulations, and/or methods (e.g., including one or more types of MSCs, either alone or in conjunction with one or more other agents) disclosed herein.

The term “topical administration” refers to a type of non-invasive administration to the skin, orifices, and/or mucosa of a subject. Topical administrations can be administered locally; that is, they are capable of providing a local effect in the region of application without systemic exposure. Topical formulations can, however, provide one or more systemic effects via, e.g., adsorption into the blood stream of the individual. Routes of topical administration include, but are not limited to, cutaneous and transdermal administration, buccal administration, intranasal administration, intravaginal administration, intravesical administration, ophthalmic administration, pulmonary administration, and rectal administration.

The terms “treating,” “treatment,” and “therapy” refer, either individually or in any combination, to any success or indicia of success in the attenuation or amelioration of an injury, disease, symptom, disorder, pathology, and/or condition, and/or pathological condition, including any objective or subjective parameter such as, for instance, abatement, remission, diminishing of symptoms or making the condition more tolerable to the patient, slowing the rate of degeneration or decline, making the final point of degeneration less debilitating, improving a subject's physical or mental well-being, and/or prolonging the length of survival. Treatment does not necessarily indicate complete eradication or cure of the injury, disease, symptom, disorder, pathology, and/or condition, and/or pathological condition, or any associated symptom(s) thereof. The treatment may be assessed by one or more objective or subjective parameters, including, for example, the results of a physical examination, blood and other clinical tests (e.g., imaging), and the like. In at least one example, treatment with the disclosed one or more agents, formulations, and/or methods (e.g., including one or more types of MSCs, either alone or in conjunction with one or more other agents) results in a decrease in the number, volume, and/or weight of a tumor and/or metastases.

The term “molecular weight” generally refers to the relative average chain length of a bulk polymer or protein, unless otherwise specified. In practice, molecular weights can be estimated or characterized using various methods including, for example, gel permeation chromatography (GPC) or capillary viscometry. GPC molecular weights are reported as the weight-average molecular weight (MW), as opposed to the number-average molecular weight (MN). Capillary viscometry provides estimates of molecular weight as the inherent viscosity determined from a dilute polymer solution using a particular set of concentration, temperature, and solvent conditions.

Further, unless otherwise noted, technical terms are generally used according to conventional usage. Aspects of the disclosed methods employ, unless indicated specifically to the contrary, conventional methods of chemistry, biochemistry, organic chemistry, molecular biology, microbiology, recombinant DNA techniques, genetics, immunology, and/or cell biology, many of which are described below solely for the purpose of illustration. Such techniques are explained fully in technical literature sources. General definitions of common terms in the aforementioned fields, including, for instance, molecular biology, may be found in references such as, e.g., Krebs et al., Lewin's Genes X, Jones & Bartlett Learning (2009) (ISBN 0763766321); Rédei, Encyclopedic Dictionary of Genetics, Genomics, Proteomics and Informatics (3rd ed.), Springer (2008) (ISBN: 1402067532); Ausubel et al., Current Protocols in Molecular Biology, John Wiley and Sons (updated July 2008) (ISBN: 047150338X); Ausubel et al., Short Protocols in Molecular Biology: A Compendium of Methods from Current Protocols in Molecular Biology (2nd ed.), Wiley-Interscience (1989) (ISBN 0471514705); Glover, et al., DNA Cloning: A Practical Approach, Vol. I-II, Oxford University Press (1985) (ISBN 0199634777); Anand et al., Techniques for the Analysis of Complex Genomes, Academic Press (1992) (ISBN 0120576201); Hames et al., Transcription and Translation: A Practical Approach, Oxford University Press (1984) (ISBN 0904147525); Perbal et al., A Practical Guide to Molecular Cloning (2nd ed.), Wiley-Interscience (1988) (ISBN 0471850713); Kendrew et al., Encyclopedia ofMolecular Biology, Wiley-Blackwall (1994) (ISBN 0632021829); Meyers et al., Molecular Biology and Biotechnology: A Comprehensive Desk Reference, Wiley-VCH (1996) (ISBN 047118571X); Harlow et al., Antibodies: A Laboratory Manual, Cold Spring Harbor Laboratory Press (1988) (ISBN 0879693746); Coligan et al., Current Protocols in Immunology, Current Protocols (2002) (ISBN 0471522767); Annual Review of Immunology; articles and/or monographs in scientific journals (e.g., Advances in Immunology); and other similar references.

Anti-Tumor Immunity

The term “anti-tumor immunity,” at least as used herein, refers to the innate and/or adaptive immune response elicited against one or more tumor antigens. Such “tumor antigens” refers to antigens that tumors generate, express, and/or release into their surrounding environment. This environment may be referred to herein as the “tumor microenvironment.” As part of the immune response to tumors, dendritic cells (“DC” or “DCs”) engulf and process these tumor antigens.

The DCs then present one or more portions of the tumor antigens within major histocompatibility class (“MHC”) molecules to naïve CD4+ and CD8+ T lymphocytes. Major histocompatibility class (also referred to as “major histocompatibility complex”) molecules are cell surface proteins expressed by various immune cells, including, for instance, the aforementioned T lymphocytes. Such T lymphocytes (also referred to variously as “T cells” or “thymocytes”) are a type of white blood cell; accordingly, they are a part of the immune system/immune response and develop from stem cells. CD4+ T lymphocytes are those cells that express (i.e., are “positive” for, hence the “+” designation) the glycoprotein CD4 (“cluster of differentiation 4”). Similarly, CD8+ T lymphocytes are those cells that express the glycoprotein CD8 (“cluster of differentiation 8”).

Once naïve CD4+ and CD8+ T lymphocytes bind to the one or more portions of the tumor antigen displayed on the surface of DCs, such lymphocytes activate, proliferate, and differentiate into CD4+ T helper cells (also referred to as “helper T cells” or “CD4+ Th cells”) and CD8+ cytotoxic T lymphocytes (also referred to as “killer T cells,” “CD8+ CTLs,” or “CTLs”), respectively. These differentiated cells help to perform various immune system functions, including, for instance, immune-mediated cell death, a process in which the immune system triggers cell death in response to, for example, an infected cell or a cancer cell.

Specifically, CD4+ Th cells orchestrate an anti-tumor immune response through production of various factors and/or biological compounds, including, for instance, interleukin (IL)-2. IL-2 increases the proliferation of CD8+ CTLs and secretes interferon gamma (IFN-γ), which induces generation of the anti-tumorigenic M1 phenotype in tumor-infiltrated macrophages (“TAM” or “TAMs”). TAMs are cancer stromal cells that play a role in a tumor development and/or progression. Two phenotypes or subsets of TAMs are the aforementioned M1 phenotype and the M2 phenotype.

The M1 phenotype is referred to herein as “M1 macrophages.” M1 macrophages generally activate anti-tumor mechanisms and/or pathways. For instance, M1 macrophage-derived compounds (e.g., chitinases and proteases) can lyse tumor cells, while M1 macrophage-sourced chemokines can attract CD8+ CTLs and natural killer (“NK”) cells in the tumor microenvironment. By contrast, the M2 phenotype, referred to herein as “M2 macrophages,” can generally activate one or more aspects of tumor progression. Normal functions of M2 macrophages include, for instance, assisting in repair processes (e.g., tissue repair). Accordingly, M2 macrophages can promote tumor growth by, for instance, releasing repair and/or growth factors.

NK cells are lymphocytes that are related to B cells and T cells and come from the same progenitor as those cells. NK cells perform a variety of immune system functions, including destroying cells that have been infected. Additionally, NK cells may play a role in protecting against other diseases, including cancer and tumor formation. Mature NK cells in humans can be divided into two different subsets, depending on the relative density of cluster of differentiation 56 (CD56) on the surface of these cells. These subsets are referred to as CD56^{bright} and CD56^{dim}; the former are common in secondary lymphoid tissues, while the latter are common in peripheral blood. Further, CD56^{bright} cells may give rise to CD56_{dim} cells.

CTLs and NK cells share various common effector mechanisms for eliminating cancer cells, including, for instance, granule exocytosis and the death ligand/death receptor system. For instance, programmed death ligand-1 (PD-L1) is a molecule, expressed by T cells, that may be upregulated on the surface of tumor cells. PD-L1 can bind to programmed death (PD) receptors (e.g., PD-1 receptor), which can be expressed on various lymphocytes. This mechanism can result in immune system evasion.

Further, perforin sourced from CTLs and/or NK cells can form pores in the membranes of tumor cells, allowing various compounds (e.g., granzyme B) to access the cytosol of such tumor cells, inducing apoptosis. Such apoptosis results from the cleavage of important intracellular substrates that control the survival of the tumor cells.

Additionally, CTLs and NK cells can express specific cell death ligands, such as, for example, programmed death ligands (e.g., PD-L1 and PD-L2) and Fas ligand (FASL), which activate extrinsic and/or intrinsic mitochondrial apoptotic pathways in malignant cells (e.g., tumor cells). This can occur, for example, through the binding to PD and Fas receptors that are expressed on the membranes of such malignant cells.

Various other cells work in opposition to M1 macrophages, CTLs, and/or NK cells. Such cells include, for instance, immunosuppressive CD4+ FOXP3+ T regulatory cells (“Treg” or “Tregs”), tumor-associated M2 macrophages, N2 neutrophils, and myeloid-derived suppressor cells (“MDSC” or “MDSCs”). These cells generally promote tumor growth and progression.

Tregs are regulatory T cells (also referred to as “suppressor T cells”) that are generally immunosuppressive and can, for instance, help to prevent autoimmune diseases. Tregs can express several biomarkers, such as, for example, CD4 and forkhead box P3 (FOXP3). FOXP3 (also referred to as “scurfin”) is a protein that assists in regulation of regulatory pathways, including, for example, development of Tregs. Thus, the aforementioned CD4+ FOXP3+ T regulatory cells are positive for (i.e., express) both CD4 and FOXP3.

N2 neutrophils are a subset of neutrophils (also referred to as “neutrocytes” or “heterophils”), which are granulocytes that are formed in the bone marrow. N2 neutrophils may function in immunosuppression and promote the development and/or growth of tumors (e.g., angiogenesis and metastases). These neutrophils can secrete various factors and/or compounds, including, for example, hepatocyte growth factor (HGF), reactive oxygen species (ROS), and matrix metalloproteinase (MMPs).

MDSCs are a group of immune cells that are derived from myeloid cells, which are themselves cells that originate from stem cells. MDSCs can have immunosuppressive properties and can proliferate under abnormal conditions (e.g., cancer). Notably, MDSCs are present in many cancer patients, and may exhibit their immunosuppressive properties by producing various biological compounds, including, for example, arginase, ROS, nitric oxide synthase, and IL-10. Additionally, MDSCs can interact with other immune cells, including, for example, T cells, DCs, macrophages (also denoted “My”), and NK cells. Specifically, MDSCs can block T-cell activation by consuming cysteine and/or limiting available cysteine for T cells. Cysteine is an important amino acid in the T-cell activation process since T cells lack cystathionase, an enzyme that converts the amino acid methionine to cysteine. Further, T cells cannot import the amino acid cystine and convert it to cysteine.

Of the aforementioned immunosuppressive cell types, both Tregs and MDSCs express, among others, cytotoxic T-lymphocyte-associated protein 4 (CTLA-4) and PD-L1. Moreover, they produce specific immunosuppressive cytokines (e.g., IL-10, transforming growth factor beta (TGF-β)) that inhibit proliferation, activation, and/or effector functions of CTLs and NK cells. Further, M2 macrophages and N2 neutrophils can secrete pro-angiogenic factors (e.g., vascular endothelial growth factor (VEGF), TGF-β, prostaglandin E2 (PGE2)), which induce the generation of new blood vessels. Such blood vessel growth can enable enhanced tumor growth and progression.

Since different immune cells affect tumor growth in opposite directions, modification of immune cell phenotypes and/or functions can be used in different immunotherapeutic treatments (e.g., cancer treatments).

MSC-Dependent Suppression of Anti-Tumor Immunity

MSCs are self-renewable, multipotent stem cells that are “plastic,” a term which, at least as used herein, means that MSCs are capable of exhibiting adaptability in response to one or more changes and/or alterations in their environment. As a non-limiting example, MSCs can adapt their phenotype and/or function in response to certain characteristics (e.g., the cytokine profile) of neighboring cells, including, for instance, tumor and/or cancer cells.

MSCs can be derived from multiple sources within the human body, including, for instance, bone marrow (also referred to as “BM-MSC” or “BM-MSCs”), adipose tissue (also referred to as “AT-MSC” or “AT-MSCs”), muscles, skin, the placenta, the femoral shaft, the iliac crest, umbilical cord blood, the umbilical cord itself, Wharton's jelly, the endometrium, menstrual blood, dental pulp, periodontal ligaments, the gingiva, apical papilla, and dental follicles.

MSCs can play a role in various immune responses. For example, after injury, alarmins, endogenous molecules released from damaged cells, activate tissue-resident MSCs, which express PD-L1 and produce various immunoregulatory factors that modulate the cytokine milieu of the local environment. This can alter the phenotype and function of different immune cells.

MSCs can also affect the antigen-presenting properties of immune cells, including, for example, DCs, B cells, and macrophages. Additionally, MSCs can modulate the phagocytic ability of neutrophils and monocytes, change the polarization of macrophages, modify the cytotoxic properties of NK cells, and regulate the proliferation, activation and/or effector functions of CD4+ and CD8+ T cells.

Additionally, MSCs, via juxtracrine and/or paracrine signaling, can induce the generation and/or expansion of immunosuppressive Tregs and MDSCs, which results in alleviation of ongoing inflammation.

Juxtracrine signaling (also referred to as “contact-dependent signaling”) is a type of intercell signaling that requires close contact. Such signaling can occur when a ligand on one surface binds to a receptor on another adjacent surface. There are at least three different types of juxtracrine signaling, including, for instance (1) interaction between a membrane compound (e.g., lipid) of a first cell and a membrane protein of a second, nearby cell, (2) junctions between two nearby cells that permit passage of specific molecules, and (3) interaction between a membrane protein of a first cell and a biological compound in the extracellular matrix. Further, juxtracrine signaling can occur for specific growth factors and cytokines, including growth factors that play a role in the immune response.

Paracrine signaling is another type of intercell signaling in which a given cell produces one or more signals, thereby inducing a change in one or more nearby cells. Paracrine signaling can proceed via specific paracrine factors, which diffuse over the distance between the given cell and the one or more nearby cells. Thus, a cell engaging in paracrine signaling can produce, and excrete, the aforementioned paracrine factors into the extracellular matrix. Many paracrine factors bind to specific receptors (e.g., receptors in the TGF-β family).

Regulation of MSCs can proceed via several pathways and biological factors, including, for example, various cytokines, transcription factors, and nucleic acids. For instance, transcription factors such as Runt-related transcription factor 2 (Runx2), SRY-related high-mobility group-box gene 9 (Sox9), peroxisome proliferation-activated receptor γ (PPARγ), various members of the helix-loop-helix family transcription factors (e.g., myoblast determination protein 1 (MyoD), and various members of the GATA zinc finger transcription factor family (e.g., GATA4, GATA6) can play a role in MSC differentiation.

Since MSCs represent an important cellular constituent of the tumor microenvironment and can modulate the phenotypes and/or functions of immune cells that participate in anti-tumor immune responses, MSCs can be used for immunotherapies in the treatment of malignant diseases (e.g., cancer).

MSC Compositions

In at least one embodiment of the present disclosure, one or more compositions are disclosed that comprise one or more types of MSCs and/or one or more biological compounds extracted and/or derived therefrom (e.g., anti-tumor proteins, cytokines, etc.) (also referred to herein as “MSC Composition” or “MSC Compositions”). The MSCs may be derived from various sources within the human body and/or subject, including, for example, bone marrow (also referred to as “BM-MSC” or “BM-MSCs”), adipose tissue (also referred to as “AT-MSC” or “AT-MSCs”), muscles, skin, the placenta, the femoral shaft, the iliac crest, umbilical cord blood, the umbilical cord itself, Wharton's jelly, the endometrium, menstrual blood, dental pulp, periodontal ligaments, the gingiva, apical papilla, and dental follicles. In at least an additional embodiment, other active agents may be co-administered with one or more MSC Compositions, including, for example, secondary anti-cancer agents, anti-inflammatories, exogenous immune cells, small molecules, therapeutic proteins, and the like. Non-limiting examples include chemotherapeutic compounds and/or drugs, exosomes derived from MSCs, and microribonucleic acids (miRNA), each of which will be discussed further below. In at least one embodiment, the MSC Compositions retain most, if not all, of the biological compounds (including anti-tumor compounds) after short-term or long-term storage under temperature-controlled conditions. The MSC Compositions may be stored under any such conditions known in the art, e.g., as a liquid, as a lyophilized powder, etc. The total protein content of the MSC Compositions when compared to MSCs extracted from a subject is, for example, at least 60%, 70%, 80%, and preferably more than 85%.

In at least one embodiment, one or more of the MSC Compositions disclosed herein exhibits any of the aforementioned anti-tumor effects when administered into a subject, including, for instance, (1) altering the phenotype and/or function of various immune cells, (2) modulating the phagocytic ability of neutrophils and/or monocytes, (3) changing the polarization of macrophages, (4) modifying the cytotoxic properties of NK cells, (5) regulating the proliferation, activation and/or effector functions of CD4+ and CD8+ T cells, (6) inducing the generation and/or expansion of immunosuppressive Tregs and MDSCs, and (7) any other anti-tumor effect described herein.

In at least an additional embodiment, one or more of the MSC Compositions are not heat-treated, chemical-treated, or fractionated to produce any of the formulations described herein. In at least one embodiment, one or more formulations that include one or more of the MSC Compositions (also referred to “MSC Formulations”) retain more than 50%, more than 60%, more than 70%, more than 80%, or preferably more than 90%, of the biological compounds (including anti-tumor compounds) present in MSCs freshly extracted from a subject. In at least a further embodiment, one or more MSC Formulations are not diluted with any additional solution. In at least another embodiment, one or more MSC Formulations are not concentrated.

In at least one embodiment, one or more MSC Compositions and/or one or more MSC Formulations can be stored for long periods of time, allowing for a variety of modes of application, including distribution and storage as aerosols, solutions, powders, etc. In at least an additional embodiment, one or more MSC Compositions and/or one or more MSC Formulations are refrigerated at about 1° C. to about 10° C. for long-term storage. In at least a further embodiment, the one or more MSC Compositions and/or one or more MSC Formulations are refrigerated at 4° C. for up to 12 months or more. Preferably, long-term storage does not reduce the quantity and/or quality of the total soluble proteins and/or biological compounds present. For at least one embodiment, the total soluble proteins and/or biological compounds retained after long-term storage in refrigerated conditions is about 10%, 20%, 30%, 40%, 50%, 60%, 70%, 80%, or 90% relative to MSCs extracted from a given subject.

In at least one embodiment, one or more MSC Formulations can be supplied as a clear one-part solution in a suitable container for storage at 4° C., or for storage at −20° C., or at −80° C. As non-limiting examples, liquid formulations in prefilled aliquots can be suitable for storage at 1-5° C., or for storage at −20° C., or at −80° C. The liquid formulation can be suitable for topical application in, e.g., a nebulizer or an inhaler. In at least an additional embodiment, the fluid can be supplied as a kit that can be stored at 4° C., at −20° C., or at −80° C. until needed.

In at least one embodiment, one or more MSC Formulations use a final filtration through a 0.2 μm filter. In at least an additional embodiment, such filtration is necessary to optimize sterile conditions without the requirement for irradiation (e.g., e-beam treatment). In at least a further embodiment, the one or more MSC Formulations have a 10′ sterility assurance level without irradiation. In at least another embodiment, lyophilisate versions of the one or more MSC Formulations may also be irradiated by e-beam irradiation or gamma ray irradiation to fully sterilize the lyophilisate.

In at least one embodiment, one or more MSC Compositions and/or one or more MSC Formulations comprise various growth factors. Non-limiting examples of such growth factors include TGF-β, VEGF, and others as described further below.

In at least one embodiment, one or more MSC Compositions and/or one or more MSC Formulations are administered to a subject in combination with one or more additional therapeutic, diagnostic, and/or prophylactic agents to alleviate pain (e.g., pain associated with one or more types of cancer), facilitate healing, and/or to reduce or inhibit scarring. In at least an additional embodiment, one or more MSC Compositions comprise one or more additional compounds to prevent or treat cancers and tumors, and/or to relieve symptoms such as inflammation. Non-limiting examples include antimicrobial agents, analgesics, local anesthetics, anti-inflammatory agents, antioxidants, immunosuppressants, anti-allergenic agents, enzyme cofactors, essential nutrients, and growth factors.

In at least one embodiment, one or more MSC Compositions and/or one or more MSC Formulations are administered to a subject for prevention or treatment of cancer and/or a tumor (e.g., a cancerous or non-cancerous tumor). In one example, an effective amount of one or more MSC Formulations are administered adjacent to a site in need thereof. In at least another embodiment, one or more MSC Compositions and/or one or more MSC Formulations are administered with a second cancer therapeutic (e.g., chemotherapy, humanized molecular antibody, etc.) to a subject for prevention or treatment of cancer and/or a tumor. Accordingly, in at least one embodiment, one or more MSC Compositions and/or one or more MSC Formulations may be considered a targeted adjuvant therapy, serving to complement traditional cancer therapeutic approaches (e.g., chemotherapy) while minimizing adverse side effects. Additional secondary therapeutic agents include, but are not limited to, antibiotics, cytokines, and growth factors (e.g., fibroblast growth factor, hepatocyte growth factor, cell-cycle checkpoint inhibitors, platelet-derived growth factor, vascular endothelial cell growth factor, and insulin-like growth factor). In at least another embodiment, secondary therapeutic agents include, for instance, hyaluronic acid or glycosaminoglycans.

In at least one embodiment, additional active agents may be administered with one or more MSC Compositions and/or one or more MSC Formulations, the active agents including, for instance, small molecules, biomolecule, peptides, sugar, glycoproteins, polysaccharides, lipids, nucleic acids, and/or combinations thereof. Suitable small molecule active agents include, but are not limited to, organic and organometallic compounds. In at least one instance, the aforementioned small molecule active agent has a molecular weight of less than about 2000 g/mol, more preferably less than about 1500 g/mol, and most preferably less than about 1200 g/mol. The small molecule active agent can be a hydrophilic, hydrophobic, or amphiphilic compound. In at least one example, one or more additional agents may be dispersed, dissolved, and/or suspended in one or more MSC Compositions and/or one or more MSC Formulations.

Volume of administration of one or more MSC Compositions and/or one or more MSC Formulations is tissue-specific and dependent on the stage of the disease or disorder. Dosages can be readily determined by those of skill in the art. See, e.g., Ansel et al., Pharmaceutical Dosage Forms and Drug Delivery Systems (6th ed.), Williams and Wilkins (1995). Additionally, one or more MSC Compositions and/or one or more MSC Formulations may be administered in conjunction with other types of cells, e.g., other exogenous stem cells, pluripotent cells, somatic cells, and/or combinations thereof. In at least one embodiment, one or more therapeutic, prophylactic, and/or diagnostic agents is administered prior to, in conjunction with, and/or subsequent to treatment with one or more MSC Compositions and/or one or more MSC Formulations.

In at least one embodiment, the aforementioned therapeutic, prophylactic and/or diagnostic agents may be administered in a neutral form, or in the form of a pharmaceutically acceptable salt. In at least one example, it may be desirable to prepare a formulation containing a salt of an agent due to one or more of the salt's advantageous physical properties, such as, for example, enhanced stability, a desirable solubility, and/or a desirable dissolution profile.

In at least one embodiment, pharmaceutically acceptable salts are prepared by reaction of the free acid or base forms of an active agent with a stoichiometric amount of the appropriate base or acid in water or in an organic solvent, or in a mixture of the two; generally, non-aqueous media such as, for example, ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts, as well as salts formed by reaction of the drug with a suitable organic ligand (e.g., quaternary ammonium salts). Lists of suitable salts are found, for example, in Adej are et al., Remington: The Science and Practice of Pharmacy (23rd ed.), Academic Press (2020).

In at least one embodiment, the aforementioned secondary agent administered with one or more MSC Compositions and/or one or more MSC Formulations comprises a diagnostic agent such as, for example, paramagnetic molecules, fluorescent compounds, magnetic molecules, and radionuclides, X-ray imaging agents, and/or contrast media.

In at least one embodiment, one or more MSC Formulations comprises one or more local anesthetics. Non-limiting examples of such local anesthetics include tetracaine, lidocaine, amethocaine, proparacaine, lignocaine, and bupivacaine. In at least one example, one or more additional agents, such as, e.g., a hyaluronidase enzyme, is also added to the one or more MSC Formulations to accelerate and/or improve dispersal of the local anesthetic.

In at least one embodiment, one or more MSC Compositions and/or one or more MSC Formulations are used in combination with one or more antimicrobial agent. An antimicrobial agent, at least in the context of the present disclosure, is a substance that inhibits the growth of microbes including, for instance, bacteria, fungi, viruses, and/or parasites. Accordingly, antimicrobial agents include, for example, antiviral agents, antibacterial agents, antiparasitic agents, and anti-fungal agents. Non-limiting examples of antiviral agents include, e.g., ganciclovir and acyclovir. Non-limiting examples of antibiotic agents include, for example, aminoglycosides (e.g., streptomycin, amikacin, gentamicin, and tobramycin), ansamycins (e.g., geldanamycin and herbimycin), carbacephems, carbapenems, cephalosporins, glycopeptides (e.g., vancomycin, teicoplanin, and telavancin), lincosamides, lipopeptides (e.g., daptomycin, macrolides such as azithromycin, clarithromycin, dirithromycin, and erythromycin), monobactams, nitrofurans, penicillins, polypeptides (e.g., bacitracin, colistin, and polymyxin B), quinolones, sulfonamides, and tetracyclines.

Other exemplary antimicrobial agents include, for instance, iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

In at least one embodiment, one or more MSC Compositions and/or one or more MSC Formulations are administered in combination with one or more local anesthetics. A local anesthetic, at least in the context of the present disclosure, is a substance that causes reversible local anesthesia and has the effect of loss of sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and combinations thereof. In at least another aspect of this embodiment, the one or more MSC Compositions and/or one or more MSC Formulations include an anesthetic agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition and/or formulation.

In at least one embodiment, one or more MSC Compositions and/or one or more MSC Formulations are administered in combination with one or more anti-inflammatory agents. Anti-inflammatory agents reduce inflammation and include, for instance, steroidal and non-steroidal drugs. Suitable steroidal active agents include, for example, glucocorticoids, progestins, mineralocorticoids, and corticosteroids. Other non-limiting examples of anti-inflammatory agents include triamcinolone acetonide, fluocinolone acetonide, prednisolone, dexamethasone, loteprendol, fluorometholone, ibuprofen, aspirin, and naproxen. Non-limiting examples of immune-modulating drugs include cyclosporine, tacrolimus, and rapamycin. Non-limiting examples of non-steroidal anti-inflammatory drugs (NSAIDs) include ketorolac, nepafenac, and diclofenac. In at least one embodiment, anti-inflammatory agents are anti-inflammatory cytokines. Non-limiting examples of such cytokines include IL-10, IL-17, TNF-α, TGF-β, IL-35, and others described below.

In at least one embodiment, one or more MSC Compositions and/or one or more MSC Formulations are administered in combination with one or more growth factors. As mentioned above herein, growth factors are proteins and/or glycoproteins capable of stimulating cellular growth, proliferation, and/or cellular differentiation. Non-limiting examples of growth factors include transforming growth factor beta (TGF-β), transforming growth factor alpha (TGF-α), granulocyte-colony stimulating factor (GCSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF8), growth differentiation factor-9 (GDF9), acidic fibroblast growth factor (aFGF or FGF-1), basic fibroblast growth factor (bFGF or FGF-2), epidermal growth factor (EGF), vascular endothelial growth factor (VEGF), and hepatocyte growth factor (HGF).

In at least one embodiment, one or more MSC Compositions and/or one or more MSC Formulations are administered in combination with one or more enzyme cofactors, and/or one or more essential nutrients. Non-limiting examples of such cofactors include vitamin C, biotin, vitamin E, and vitamin K. Non-limiting examples of such essential nutrients include amino acids, fatty acids, etc.

In at least one embodiment, one or more MSC Compositions and/or one or more MSC Formulations comprise at least one eukaryotic cell type other than one or more types of MSCs. Non-limiting examples of such eukaryotic cell types include non-mesenchymal stem cells, immune cells (e.g., T lymphocytes, B lymphocytes, natural killer cells, macrophages, dendritic cells), and combinations thereof. In at least an additional embodiment, the cells used are cells that dampen one or more inflammation responses (e.g., regulatory T cells). In at least a further embodiment, exosomes are generated ex vivo from one or more types of MSCs.

MSC Formulations

In at least one embodiment, one or more of the MSC Formulations are packaged into sterile dosage units, which can be stored and distributed for use by attending physicians and/or other healthcare professionals. These formulations, which may be in various forms (e.g., fluid, lyophilized), can be administered through, for instance, sterile packaged syringes for injection, dropper bottles, tubes, or vials of solution. The dosages for injectables generally will be 0.1 cubic centimeter (cc), 0.25 cc, 0.5 cc, 1.0 cc, 2 cc, 5 cc, 10 cc, and 20 cc. The injectables can be administered at, for example, the site of the tumor. In at least one embodiment, one or more formulations described herein are sprayed onto, soaked into, or powder-dispersed onto the tumor site or cancer lesion. Efficacy of administration can generally be determined by, for instance, physician evaluations, patient self-evaluations, and/or quality of life evaluations.

In the aforementioned at least one embodiment, the sterile one or more MSC Formulations can be administered in concentrated form, diluted with sterile water or buffer, or formulated as a solution or suspension. The one or more MSC Formulations may be administered with additional therapeutic, prophylactic, and/or diagnostic agents, either in solution or suspension, or as particles (e.g., nanoparticles, liposomes, microparticles), or directly at tumor sites.

Non-limiting examples of excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS) and may be administered to an individual without causing undesirable biological side effects or unwanted interaction(s).

In at least one embodiment, one or more MSC Formulations are in a solution or suspension. In at least one embodiment, the solutions may include sterile filtered liquids, diluted liquids, buffers, lipids, and/or oils. Emulsions are generally dispersions of oily droplets in an aqueous phase. In at least one example, there should be no evidence of breaking or coalescence in an emulsion. Suspensions generally contain solid particles dispersed in a liquid vehicle; in at least another example, such suspensions must be homogeneous when shaken gently and remain sufficiently dispersed to enable the correct dose to be removed from the container. A sediment may occur, but this should disperse readily when the container is shaken, and the size of the dispersed particles should be controlled. The active ingredient and any other suspended material must be reduced to a particle size small enough to prevent irritation and damage to the site of administration. Suspensions may comprise suitable additives, such as, for instance, antimicrobial agents, antioxidants, and stabilizing agents. In at least one embodiment, when the solution is dispensed in a multidose container that is to be used over a period of time longer than 24 hours, a preservative must be added to ensure microbiologic safety over the period of use.

In at least one embodiment, the aforementioned solution or suspension is physiological, for example, at pH 7.4. In at least an additional embodiment, the pH is optimized for both stability of the active pharmaceutical ingredient and physiological tolerance. If a buffer system is used, it must not cause precipitation or deterioration of the active ingredient. The normal useful pH range is 6.5 to 8.5, although lower pHs may be used. Buffers and/or pH adjusting agents or vehicles can be added to adjust and stabilize the pH at a desired level. In at least a further embodiment, one or more such buffers are included to minimize any change in pH during storage. Changes in pH can affect the solubility and stability of drugs; consequently, it is important to minimize fluctuations in pH. The buffer system should be sufficient to maintain the pH throughout the expected shelf-life of the product. Low concentrations of buffer salts are used to prepare buffers of low buffer capacity.

Aqueous solution preparation can be optimized and/or supplemented for isotonicity, pH, antimicrobial agents, antioxidants, and/or viscosity-increasing agents. Solutions are generally considered isotonic when the tonicity is equal to that of a 0.9% solution of sodium chloride (NaCl). Tissues can usually tolerate solutions equivalent to 0.5-2% of sodium chloride. Solutions that are isotonic are therefore preferred. An amount equivalent to 0.9% NaCl is used in at least one embodiment. In at least a further embodiment, hypertonic solutions are prepared to facilitate solubility of one or more other agents co-administered with the one or more MSC Compositions and/or one or more MSC Formulations. A widely used buffer solution is Sorensen's modified phosphate buffer, which is generally used to modulate pH values between the range of 6.5-8.0. This buffer comprises two stock solutions, one acidic containing NaH2PO4, and one basic containing Na2HPO4. Other suitable buffers known in the art include, for example, acetate, borate, carbonate, citrate, and phosphate buffers.

In at least one embodiment, one or more MSC Formulations are packaged and/or distributed in liquid form. Alternatively, one or more such formulations can be packed as a solid, which can be obtained by, for example, lyophilization of a suitable liquid formulation. In at least an additional embodiment, the solid can be reconstituted with an appropriate carrier or diluent prior to administration. Solutions, suspensions, and/or emulsions for administration to a subject may also contain one or more tonicity agents to adjust the isotonic range of the formulation. Suitable tonicity agents are known in the art, non-limiting examples of which include glycerin, mannitol, sorbitol, sodium chloride, and other electrolytes.

Solutions, suspensions, aerosols, sprays, and/or emulsions may also contain one or more preservatives to prevent contamination (e.g., bacterial contamination). Suitable preservatives are known in the art, non-limiting examples of which include polyhexamethylenebiguanidine (PHMB), benzalkonium chloride (BAK), stabilized oxychloro complexes (otherwise known as) PURITE®, phenylmercuric acetate, chlorobutanol, sorbic acid, chlorhexidine, benzyl alcohol, parabens, thimerosal, and combinations and/or mixtures thereof.

Solutions, suspensions, and/or emulsions may also contain one or more excipients known in the art, non-limiting examples of which include dispersing agents, wetting agents, and suspending agents.

In at least one embodiment, one or more MSC Compositions and/or one or more MSC Formulations are provided in a kit. Specific formulations can be prepared using a pharmaceutically acceptable “carrier” composed of materials that are considered safe and effective and may be administered to an individual without causing undesirable side effects. These formulations (e.g., in lyophilized or fluid form) can be in sterile packaged syringes for injection, and/or tubes or jars of solution. The dosages for the injectables can be 0.1 cc, 0.25 cc, 0.5 cc, 1.0 cc, 2 cc, 5 cc, 10 cc, and 20 cc. Typically sterile kits also comprise at least one liquid to rehydrate any dry components. The kit may also include various elements facilitating the administration of prophylactics or treatments of cancer, tumors, and other disorders, such as, for example, syringes and one or more applicators (e.g., needles).

Methods of Administration

Methods of using and/or administering one or more MSC Compositions and/or one or more MSC Formulations to a subject for therapeutic, diagnostic, and/or prophylactic applications, especially with respect to cancers, tumors, and other related disorders are further disclosed herein.

In at least one embodiment, one or more MSC Compositions and/or one or more MSC Formulations are administered to a mammalian subject (e.g., terrestrial mammal, aquatic mammal, and the like). Such administration is performed using a suitable dosing regimen, as described above herein, and for a period of time effective to prevent formation of tumors and/or to promote healing, repair, and/or regeneration of tissues.

In at least one embodiment, one or more MSC Compositions and/or one or more MSC Formulations experience limited perfusion and therefore may be retained at the site of application and/or injection for an extended period of time. In at least an additional embodiment, after administration, the one or more MSC Compositions and/or one or more MSC Formulations remain at the site of application for at least 6 hours, at least 12 hours, at least 1 day, at least 2 days, at least 3 days, at least one week, at least 2 weeks, at least 3 weeks, at least 4 weeks, at least 5 weeks, at least 6 weeks, at least 7 weeks, at least 2 months, at least 3 months, at least 6 months, at least 9 months, or at least 1 year or more.

Methods of using one or more MSC Compositions and/or one or more MSC Formulations to prevent and/or treat cancer (e.g., blood cancers and other cancers described herein), tumors, and other disorders are described herein. In at least one embodiment, the methods, compositions, and/or formulations are effective in preventing and/or treating cancers (e.g., breast cancer) and other non-cancerous tumors. In at least an additional embodiment, the one or more MSC Compositions and/or one or more MSC Formulations are administered in one or more amounts effective to restore tissues impacted by cancer and/or tumor growth to about 10%, 20%, 30%, 40%, 50%, 60%, 70%, 80%, 90%, 100%, 150%, 200%, 250%, 300%, or more than 300% of the damage present at the time of treatment, as measured by endogenous tissue regrowth.

In at least one embodiment, one or more MSC Compositions and/or one or more MSC Formulations are administered by injection near the site of injury or tumor infarction. In at least another embodiment, one or more MSC Compositions and/or one or more MSC Formulations is sprayed onto, soaked into, and/or powder-dispersed onto the site of tumor growth.

The compositions, formulations, and/or methods of use thereof that are described herein are suitable for managing and/or treating any cancer or tumor, in addition to other associated diseases and disorders. As a non-limiting example, administration of one or more MSC Compositions and/or one or more MSC Formulations may prevent and/or treat cancer in a patient with a degenerative disease, contributing to the reduction of symptoms of both the cancer and the degenerative disease.

Additionally disclosed herein are methods of preventing and/or treating cancer (e.g., breast cancer, blood cancers, pancreatic adenocarcinomas, colorectal adenocarcinomas) via the administration of one or more MSC Compositions and/or one or more MSC Formulations. In at least one embodiment, one or more MSC Compositions and/or one or more MSC Formulations are administered to a cancer patient or a potential cancer patient in combination with radiation therapy and/or chemotherapy. In at least an additional embodiment, the methods include administering to the subject one or more MSC Compositions and/or one or more MSC Formulations in conjunction with a pharmaceutically acceptable carrier. In at least one example, the methods include administering to the subject a pharmaceutical composition including an expression vector expressing one or more co-stimulatory molecules, one or more MSC Compositions and/or one or more MSC Formulations, and a pharmaceutically acceptable carrier.

In at least one embodiment, methods of preventing tumor growth (e.g., breast cancer tumor growth) or treating a subject with a tumor include measuring a tumor sample or tumor volume from a subject, determining an appropriate dosage of one or more MSC Compositions and/or one or more MSC Formulations, and treating the subject. In at least an additional embodiment, treating the subject may include administering to the subject an effective amount of ionizing radiation in combination with an effective amount of one or more MSC Compositions and/or one or more MSC Formulations. In at least a further embodiment, one or more MSC Compositions and/or one or more MSC Formulations are administered in combination with one or more adjuvants, antigens, vaccines, allergens, antibiotics, gene therapy vectors, vaccines, kinase inhibitors, co-stimulatory molecules, Toll-like receptor (TLR) agonists, and/or TLR antagonists. In at least another embodiment, one or more MSC Compositions and/or one or more MSC Formulations are administered in combination with a second anti-cancer therapeutic agent (e.g., a chemotherapeutic nucleic acid, an immunostimulatory protein, an inflammatory molecule, an immunostimulatory molecule). In at least another embodiment, one or more MSC Compositions and/or one or more MSC Formulations are administered systemically and/or at specific tumor locations in the subject.

In at least one embodiment, a method is disclosed for treating a subject with cancer by enhancing or inducing response of cancer-associated endogenous immune cells in the subject. In at least an additional embodiment, enhancing or inducing response of cancer-associated endogenous immune cells may include administering to the subject an effective amount of one or more MSC Compositions and/or one or more MSC Formulations as a prophylactic (e.g., an amount effective at preventing the appearance and/or growth of tumors). In at least a further embodiment, enhancing or inducing response of cancer-associated endogenous immune cells may include administering to the subject an effective amount of one or more MSC Compositions and/or one or more MSC Formulations to treat a subject with cancer or a tumor. In at least another embodiment, enhancing or inducing response of cancer-associated endogenous immune cells may include administering to the subject an effective amount of ionizing radiation, then administering to the subject an effective amount of one or more MSC Compositions and/or one or more MSC Formulations, thereby enhancing or inducing the response of cancer-associated endogenous immune cells in the subject. In at least another embodiment, the cancer-associated endogenous immune cells may include, for instance, dendritic cells, macrophages, T cells, natural killer cells, and the like.

In at least one embodiment, the compositions, formulations, and/or methods of use thereof that are described herein are used to prevent and/or treat multiple cancers. In at least an additional embodiment, the one or more MSC Compositions and/or one or more MSC Formulations is administered to a subject with both cancer and another disorder (e.g., systemic inflammation, a neurodegenerative disease, etc.). A cell, tissue, or target may be a cancer cell, a cancerous tissue, harbor cancerous tissue, or be a subject or patient diagnosed or at risk of developing a disease or condition. In at least one example, a cell may be an epithelial, an endothelial, a mesothelial, a glial, a stromal, and/or a mucosal cell. The cancer cell population can include, but is not limited to, a brain, a neuronal, a blood, an endometrial, a meninges, an esophageal, a lung, a cardiovascular, a liver, a lymphoid, a breast, a bone, a connective tissue, a fat, a retinal, a thyroid, a glandular, an adrenal, a pancreatic, a stomach, an intestinal, a kidney, a bladder, a colon, a prostate, a uterine, an ovarian, a cervical, a testicular, a splenic, a skin, a smooth muscle, a cardiac muscle, and/or a striated muscle cell. In at least a further example, cancer includes, but is not limited to, astrocytoma, acute myeloid leukemia, anaplastic large cell lymphoma, acute lymphoblastic leukemia, angiosarcoma, B-cell lymphoma, Burkitt's lymphoma, breast carcinoma, bladder carcinoma, carcinoma of the head and neck, cervical carcinoma, chronic lymphoblastic leukemia, chronic myeloid leukemia, colorectal carcinoma, endometrial carcinoma, esophageal squamous cell carcinoma, Ewing's sarcoma, fibrosarcoma, glioma, glioblastoma, gastrinoma, gastric carcinoma, hepatoblastoma, hepatocellular carcinoma, Kaposi's sarcoma, Hodgkin lymphoma, laryngeal squamous cell carcinoma, larynx carcinoma, leukemia, leiomyosarcoma, lipoma, liposarcoma, melanoma, mantle cell lymphoma, medulloblastoma, mesothelioma, myxofibrosarcoma, myeloid leukemia, mucosa-associated lymphoid tissue B cell lymphoma, multiple myeloma, high-risk myelodysplastic syndrome, nasopharyngeal carcinoma, neuroblastoma, neurofibroma, high-grade non-Hodgkin lymphoma, non-Hodgkin lymphoma, lung carcinoma, non-small cell lung carcinoma, ovarian carcinoma, oesophageal carcinoma, osteosarcoma, pancreatic carcinoma, pheochromocytoma, prostate carcinoma, renal cell carcinoma, retinoblastoma, rhabdomyosarcoma, salivary gland tumor, Schwanomma, small cell lung cancer, squamous cell carcinoma of the head and neck, testicular tumor, thyroid carcinoma, urothelial carcinoma, and/or Wilms' tumor.

Other non-limiting examples of cancers include hematological malignancies such as, for example, leukemias, including acute leukemias (e.g., 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (e.g., chronic myelocytic (granulocytic) leukemia, chronic myelogenous leukemia, and chronic lymphocytic leukemia), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome, hairy cell leukemia, and myelodysplasia. The compositions, formulations, and/or methods of use thereof that are described herein are also used to treat non-small cell lung cancer (NSCLC), pediatric malignancies, cervical and other tumors caused or promoted by human papilloma virus (HPV), melanoma, Barrett's esophagus (pre-malignant syndrome), adrenal and skin cancers, and auto-immune, neoplastic cutaneous diseases.

Non-limiting examples of solid tumors include sarcomas (e.g., fibrosarcoma, myxosarcoma, liposarcoma, chondrosarcoma, osteogenic sarcoma, and other sarcomas), synovioma, mesothelioma, Ewing sarcoma, leiomyosarcoma, rhabdomyosarcoma, colon cancer, colorectal cancer, peritoneal cancer, esophageal cancer, pancreatic cancer, breast cancer (including basal breast carcinoma, ductal carcinoma, and lobular breast carcinoma), lung cancer, ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms' tumor, cervical cancer, fallopian tube cancer, testicular tumor, seminoma, bladder cancer (such as renal cell cancer), melanoma, and central nervous system (CNS) tumors (e.g., a glioma, glioblastoma, astrocytoma, medulloblastoma, craniopharyrgioma, ependymoma, pinealoma, hemangioblastoma, acoustic neuroma, oligodendroglioma, meningioma, neuroblastoma, and retinoblastoma). Solid tumors also include tumor metastases (e.g., metastases to the lung, liver, brain, and/or bone).

In at least one embodiment, tumors comprise non-cancerous tumors such as, for instance, benign soft tissue tumors. Non-limiting examples of benign soft tissue tumors include lipoma, angiolipoma, fibroma, benign fibrous histiocytoma, neurilemmoma, hemangioma, giant cell tumor of tendon sheath, myxoma, and the like. In at least an additional embodiment, one or more MSC Compositions and/or one or more MSC Formulations may be administered as a prophylactic or treatment for other non-cancerous soft tissue tumors, including fat tissue tumors (e.g., lipoblastoma, hibernoma), fibrous tissue tumors (e.g., elastofibroma, superficial fibromatosis, desmoid-type fibromatosis, and deep benign fibrous histiocytoma), muscle tissue tumors (e.g., leiomyomas, and rhabdomyoma), blood and lymph vessel tumors (e.g., hemangioma, glomus tumors, and lymphangioma), and nerve tissue tumors (e.g., neurofibroma and schwannoma).

In at least one embodiment, the methods described herein may include identifying and/or selecting a subject in need of treatment and/or a subject that would benefit from administration of one or more MSC Compositions and/or one or more MSC Formulations. In at least an additional embodiment, the subject to be treated is a mammal (e.g., a human, domestic animal, livestock, aquatic mammal, and the like).

One or more of various pharmaceutically acceptable carriers can be used with one or more MSC Compositions and/or one or more MSC Formulations described herein. As a non-limiting example, buffered saline and the like may be used with the one or more MSC Compositions and/or one or more MSC Formulations described herein. Optionally, these solutions may be sterilized prior to use. In at least one example, the one or more MSC Compositions and/or one or more MSC Formulations include pharmaceutically acceptable auxiliary substances such as, for example, pH adjusting and buffering agents, toxicity adjusting agents, and preservatives (e.g., sodium acetate, sodium chloride, potassium chloride, calcium chloride, sodium lactate, and the like). The concentration of these auxiliary substances and/or formulations can vary depending on individual differences in age, weight, tumor size, extent of metastasis, and condition of the subject (patient).

Methods related to the one or more MSC Compositions and/or one or more MSC Formulations and their use are provided. The one or more MSC Compositions and/or one or more MSC Formulations may be prepared as one or more pharmaceutical compositions (e.g., compositions or formulations in combination with a pharmaceutically acceptable buffer, carrier, diluent, and/or excipient) for use in one or more methods described herein. As a non-limiting example, methods are disclosed herein for administration of the one or more MSC Compositions and/or one or more MSC Formulations, methods for inducing and/or increasing the expansion and/or function of one or more types of immune cells (e.g., CD4+ regulatory T cells), either ex vivo or in vivo. Additionally disclosed herein are methods of inducing or increasing a population of one or more types of immune cells (e.g., DC cells, NK cells) in a subject in need thereof. The methods of treatment can include administering to a subject (e.g., a human patient) an effective amount of one or more MSC Compositions and/or one or more MSC Formulations to one or more cancerous or tumorigenic tissues in the subject.

In at least one embodiment, administration of one or more MSC Compositions and/or one or more MSC Formulations to a subject results in an increase in the proliferation and/or number of endogenous immune cells (e.g., anti-inflammatory cells). Generally, this increase is observed within days, weeks, or months after the initial treatment, with an observed increase up to 10%, 20%, 30%, 40%, 50%, 60%, 70%, 80%, 90%, 100%, 200%, 300%, 400%, 500%, or more than 500%.

Generally, MSCs can either support or suppress tumor progression since many factors can affect MSC-dependent immunomodulatory properties in the tumor microenvironment. Thus, it is important to understand both the nature of MSCs and the tumor microenvironment in which MSCs are exposed, since that microenvironment may influence whether MSCs promote or suppress tumor growth.

Role of MSCs in Promoting and Inhibiting Tumor Growth

Various different molecular mechanisms are responsible for MSC-based modulation of anti-tumor immunity, which are discussed below. Specifically, different signaling pathways can regulate the crosstalk and/or communications between MSCs, various immune cells, and tumor cells. For example, interactions between pro-inflammatory macrophages and MSCs can enhance the secretion of tumor necrosis factor-stimulated gene-6 (TSG-6), as well as enhance the production of anti-inflammatory T cells and macrophages.

Both MSCs associated with tumors (also referred to as “cancer-associated MSCs” or “CA-MSCs”) and exogenously administered MSCs can promote tumor growth. MSCs may become associated with a tumor via one or more processes in which MSCs migrate towards the tumor. Since tumors change the structure and/or composition of the tissue in which they grow, as well as the accompanying microenvironment, MSCs may become attracted to the tumor in a similar manner as MSCs respond to tissue damage. Moreover, since MSCs can play a role in inflammation and the regulation thereof, the fact that tumors can cause chronic inflammation may further result in MSCs migrating to the tumor site. Additionally, the tumor may release one or more compounds and/or factors that recruit MSCs to the tumor. These compounds may be, for instance, chemoattractants.

MSC-mediated tumor growth may proceed by one or more processes, including, for instance (1) preventing DC-dependent activation of naïve T cells, (2) inducing alternative activation of TAMs, (3) modulating cytokine production in helper T cells, (4) downregulating cytotoxicity of CTLs and NK cells, and (5) promoting generation and/or expansion of Tregs and MDSCs. Each of these will be discussed briefly below.

First, MSCs may prevent DC-dependent activation of naïve T cells. In particular, MSCs may block the ability of DCs to promote CD4+ and/or CD8+ T cell expansion, negatively impacting the immune response to tumors. This prevention of DC-dependent activation may be influenced, via paracrine signaling, by one or more biological compounds, including, for instance, IL-10 and the Signal Transducer and Activator of Transcription 3 (STAT3) protein. Specifically, IL-10 derived from CA-MSCs can inhibit the DC-induced proliferation of T cells by blocking the ability of DCs to provide cysteine to the T cells. Further, CA-MSC-derived IL-10 can induce phosphorylation of STAT3 in DCs. Phosphorylated STAT-3 can enter the nucleus of T cells and repress the interferon gamma-activated sequence (GAS), which serves as a cystathionase promoter sequence. This results in the suppression of DC-derived cysteine export to T cells. Such lack of cysteine results in reduced T cell proliferation and/or activation. Indeed, in environments without cysteine and/or are cysteine-deficient, naïve T cells can fail to develop properly and exhibit abnormal cellular structure and/or function. Further, lack of cysteine attenuates the production of IFN-γ in T cells and reduces T cell capacity to activate macrophages in an IFN-γ-dependent manner.

Additionally, the crosstalk between MSCs, M1 macrophages, and M2 macrophages is important for MSC-dependent regulation of tumor progression. For instance, MSCs exposed to condition medium derived from M1 macrophages (“MSC-CM” or “MSC-CMs”) can promote tumor growth in both (1) breast cancer cell lines (e.g., the MDA-MB-231-FLUC cell line), and (2) murine models of hepatocellular carcinoma and glioblastoma. This effect may be due, for instance, to the fact that the secretome (i.e., the totality of molecules and/or biological compounds produced by a cell and released into the extracellular matrix) produced by M1 macrophages can increase the expression of toll-like receptor 3 (TLR-3) on MSCs. TLR-3 signaling can promote the generation of an immunosuppressive MSC phenotype by, for instance, increasing the expression of inducible nitric oxide synthase (iNOS), chemokine (C-C motif) ligand 2 (CCL2), IL-6, and/or cyclooxygenase 2 (COX-2). In at least some instances, MSC-CMs can further suppress production of activated T cells, an effect which occurs in an iNOS and nitric oxide (NO)-dependent manner. Further, exposure of such MSCs to small interfering ribonucleic acids (“siRNA” or “siRNAs”) that inhibited iNOS activity and NO production resulted in downregulation of the immunosuppressive properties of the MSC-CMs. Tumor-promoting activity of MSC-CMs can be dependent on their capacity for enhanced production of, for instance, CCL2, COX-2, and IL-6. For instance, MSC-CMs, in a CCL2-dependent manner, elicited accumulation of C-C chemokine receptor type 2 (CCR2)-expressing M1 macrophages in tumors. The M1 macrophages, in turn, induced generation of an immunosuppressive MSC phenotype (referred to as the “MSC2” phenotype) in a TNF-α-dependent manner. MSC2 cells can exhibit an increased capacity for the production of, e.g., IL-6 and COX-2, resulting in the generation of an M2 phenotype in TAMs. M2 macrophages, through the increased production of immunosuppressive cytokines (e.g., IL-10, TGF-β, etc.) and/or pro-angiogenic factors (e.g., VEGF, PGE2) can enable enhanced tumor growth and/or progression.

CA-MSCs can also promote growth of certain cancers (e.g., pancreatic cancer) by inducing M2 polarization of TAMs. CA-MSCs can have a higher capacity, when compared to MSCs derived from bone marrow, for producing immunosuppressive cytokines (e.g., IL-10, TGF-β) and tumor-promoting growth factors (e.g., monocytes-colony stimulating factor (M-CSF), granulocyte-macrophage colony-stimulating factor (GM-CSF), and CCL2). Further, CA-MSCs may have increased tumor-promoting ability when compared to bone marrow-derived MSCs (also referred to as “BM-MSC” or “BM-MSCs”). For instance, CA-MSC-treated mice showed significantly enhanced growth and progression of pancreatic cancer when compared to mice treated with BM-MSCs. IL-6 and IL-10 derived from CA-MSCs also induced generation of M2 TAMs in pancreatic tissue, while CCL2 derived from CA-MSCs caused an increased influx of circulating M2 monocytes into pancreatic tumors. Further, M2 TAMs can produce IL-10 and IL-1 receptor antagonists (e.g., IL-1Ra) that enabled the generation of the MSC2 phenotype. Both M2 TAMs and MSC2 can produce immunosuppressive cytokines that downregulate the anti-tumor immune response, leading to further immune evasion and increased proliferation of cancer cells. Indeed, the increased presence of M2 TAMs may be responsible for the tumor-promoting activity of MSCs since their depletion significantly reduced tumor growth in mice treated with CA-MSCs.

M2 TAMs may further generate an anti-inflammatory tumor microenvironment that causes MSC-dependent suppression of tumor-infiltrated CD8+ CTLs. Hypoxia and inflammation, which can be generated during tumor progression, can induce the release of nucleotides (e.g., adenosine triphosphate (ATP) and/or adenosine diphosphate (ADP)) from dead cells (e.g., dead parenchymal cells). MSC2 can further express ectonucleotidases (e.g., of the CD39 and/or CD73 families), which are nucleotide metabolizing enzymes commonly displayed on plasma membranes. Such ectonucleotidases are responsible for metabolizing nucleotides (e.g., ATP and/or ADP) into nucleosides (e.g., adenosine). Adenosine in particular can exert immunosuppressive effects on immune cells (e.g., CD8+ CTLs) by binding to adenosine-specific receptors (e.g., the adenosine A2A receptor, also referred to as “ADORA2A”). MSC-based activation of the ADORA2A receptor in CTLs can result in the enhanced generation of cyclic adenosine monophosphate (cAMP), which (1) suppresses CTL proliferation, (2) attenuates the production of various anti-tumor cytokines (e.g., TNF-α, IFN-γ), and (3) inhibits release of additional molecules (e.g., perforins, granzyme B) in the CTLs.

Consistent with the above, BM-MSCs can suppress the anti-tumor properties of CTLs, which can result in the progression of specific cancers (e.g., multiple myeloma (“MM”)). Through the activation of the PD-L1/PD1 axis, BM-MSCs that express PD-L1 can induce apoptosis and inhibit exocytosis of specific compounds (e.g., perforins, granzyme B) in the CTLs of MM patients. Accordingly, using PD-L1 inhibitors can inhibit and/or eliminate BM-MSC-based suppression of CTLs. This can result in enhanced CTL-dependent elimination of tumor cells and an overall beneficial effect in treating cancer patients.

In addition to downregulating CTL toxicity, MSCs (including, for instance, CA-MSCs) can also regulate the phenotype, function and/or cytotoxic properties of tumor-infiltrated NK cells. The crosstalk between CA-MSCs and NK cells is an important factor in MSC-driven suppression of anti-tumor immunity. NK cells may recognize one or more molecules expressed on the surface of CA-MSCs, including, for instance, MHC class I polypeptide-related sequence (MICA), UL16 binding proteins (ULBPs), cluster of differentiation 112 (CD112), and/or cluster of differentiation 155 (CD155). One or more of these can serve as ligands for NK cell-activating receptors. Activated NK cells can be important in the anti-tumor immune response by, for instance, inducing apoptosis and/or inducing, via increased production of IFN-γ, generation of the immunosuppressive MSC2 phenotype in neighboring CA-MSCs.

In turn, CA-MSC2s can regulate proliferation, cytotoxicity, and cytokine production of tumor-infiltrating NK cells. MSCs can further, via juxtracrine signaling and in a contact-dependent manner, downregulate expression of various cytotoxic receptors on certain NK cells. Non-limiting examples of the aforementioned cytotoxic receptors include, for instance, NKp44 (also referred to as natural cytotoxicity triggering receptor 2 (NCR2), NKp30 (also referred to as natural cytotoxicity triggering receptor 3 (NCR3), NKG2D, which is a transmembrane protein that belongs to the NKG2 family of C-type lectin-like receptors, and DNAX accessor molecule-1 (DNAM-I), which is a glycoprotein that is expressed on many peripheral blood T lymphocytes. These receptors can be downregulated on, for instance, the CD56^{dim} subset of NK cells. MSCs can also, via paracrine signaling and in a PGE2-dependent manner, suppress IFN-γ production in the CD56^{bright} subtype of NK cells.

CA-MSCs can also influence MDSCs and Tregs. Specifically, CA-MSCs may induce generation and/or expansion of MDSCs and/or Tregs that attenuate anti-tumor immunity and support tumor growth and progression. MSCs produce various immunosuppressive molecules (e.g., Arginase-1, nitrous oxides (“NO”), TGF-β, IL-10) that inhibit the proliferation and/or activation of naïve T cells. One or more of the aforementioned molecules can also (1) induce apoptosis promote G₀/G₁ cell cycle arrest of Tₕ1 and Tₕ17 cells, (2) attenuate the cytotoxicity of CTLs and/or NK cells, (3) induce alternative activation of TAMS, and/or (4) promote expansion of Tregs. Tₕ1 and Tₕ17 cells are different subtypes of effector T cells that can develop from helper T cells. Tₕ1 cells (also referred to as “Type 1 helper T cells”) can lead to increased immune system responses via macrophages and/or CTLs. Tₕ17 cells are distinct from Tₕ1 cells due to the production of IL-17, which generally promotes inflammation.

IFN-γ, which can be derived from tumor-infiltrating Tₕ1 lymphocytes and/or NK cells, can be important for generation and immunosuppressive functions of MDSCs. Specifically, IFN-γ may induce enhanced expression of various immunoregulatory molecules (e.g., PD-L1, cluster of differentiation 40 (CD40)) on MDSCs. IFN-γ may further increase the synthesis of PGE2, 5100 calcium-binding protein A8 (S100A8), and/or S100 calcium-binding protein A9 (S100A9). This can induce, in an autocrine manner, the proliferation and/or activation of MDSCs.

Further, MSCs can promote the proliferation, and inhibit apoptosis, of MDSCs. MSCs can enhance the immunosuppressive properties of MDSCs by, for instance, increasing the production of NO and TGF-θ. Consequently, MSC-primed MDSCs may have an increased capacity to suppress T cell-driven anti-tumor immunity.

Tregs can express various immunoregulatory molecules (e.g., PD-L1, cytotoxic T-lymphocyte-associated protein 4 (CTLA4)) and produce different immunosuppressive cytokines (e.g., IL-10, IL-35, TGF-β), which inhibit the synthesis of TNF-α, IFN-γ, IL-17 in both Tₕ1 and Tₕ17 cells. Further, Tregs may be responsible for reducing the production of perforin and/or granzymes (e.g., granzyme B) in CTLs, resulting in a reduction of CTL anti-tumor properties. MSCs may induce the generation and/or expansion of Tregs in an indoleamine 2,3-dioxygenase (IDO)-dependent manner. IDO is a heme-containing enzyme normally expressed in a variety of human tissues, including, for example, the lungs and the placenta. IDO catalyzes the first step in the kynurenine (KYN) pathway, specifically the conversion of tryptophan (TRP) to N-formylkynurenine. KYN can be immunosuppressive and promote the expression of Treg lineage-defining transcription factors (e.g., FOXP3) in naïve T cells, enabling the generation of immunosuppressive CD4+ FOXP3+ Tregs in various tissues (e.g., lymph organs). Additionally, in the tumor microenvironment, MSC-sourced IDO can prevent trans-differentiation of Tregs in anti-tumorigenic, Tₕ17-like cells.

Protein kinase B (PKB) and mammalian target of rapamycin (mTOR) are elicited by the binding of tumor antigens to the T cell receptor (TCR) of Tregs. Activated PKB and mTOR can induce the generation of pro-inflammatory and/or anti-tumorigenic phenotypes in Tregs by enhancing production of various compounds (e.g., TNF-α, IL-17, IL-22). Indeed, a low level of TRP in the tumor microenvironment can activate the general control non-derepressible 2 (GCN2) kinase, which prevents phosphorylation of PKB and inhibits PKB/mTOR signaling. By converting TRP to KYN, MSC-sourced IDO induces low TRP levels, activates GCN2 kinase, and suppresses PKB/mTOR signaling in tumor-infiltrating Tregs. This prevents the Treg trans-differentiation in anti-tumorigenic Tₕ17-like cells.

CA-MSCs may further induce the generation of a regulatory phenotype in B cells as well. Regulatory B cells are a subset of B cells that can perform various functions in the tumor microenvironment, including, for instance, (1) suppressing and/or inhibiting effector T cells, (2) inducing regulatory T cells, and (3) targeting other immune cells, such as MDSCs, NK cells, and macrophages, to inhibit anti-tumor immunity. Priming B cells with CA-MSCs can also result in attenuated production of TNF-α and increased production of IL-10. In vivo, CA-MSC-dependent induction of regulatory phenotype in B cells can contribute to the creation of systemic immunosuppression, which may enable enhanced tumor growth and/or progression.

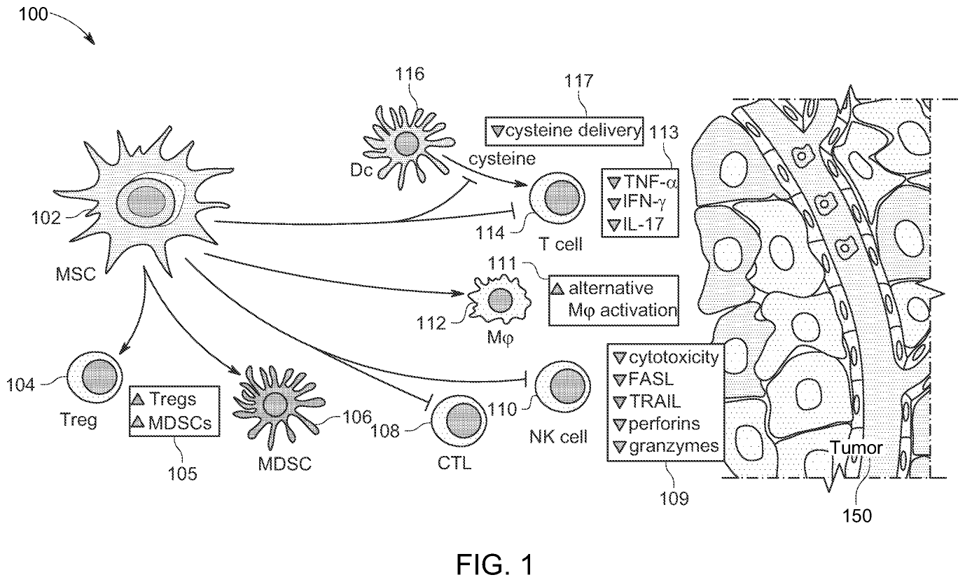

FIG. 1 shows various mechanisms 100 of MSC-mediated suppression of anti-tumor immunity. As discussed herein, MSCs 102 affect the growth, proliferation, and/or progression of tumor 150 through its interactions with Tregs 104, MDSCs 106, CTLs 108, NK cells 110, macrophages (Mφ) 112, T cells 114 (including, for instance, CTL4+ cells), and DCs 116.

Specifically, MSCs 102 can promote generation and/or expansion of Tregs 104 and MDSCs 106, as shown in block 105. Further, as shown in block 109, MSCs 102 can further block and/or inhibit one or more functions of CTLs 108 and/or NK cells 110, such as, for instance, (1) inhibiting cytotoxicity, (2) downregulating expression of FASL, (3) downregulating expression of TNF-related apoptosis-inducing ligand (TRAIL), (4) reducing secretion of perforins, and (5) reducing secretion of granzymes. MSCs may additionally cause and/or induce alternative activation of macrophages 112 (e.g., tumor-associated macrophages), as shown at block 111. Further, as shown at block 113, MSCs can suppress and/or inhibit production of, for instance, TNF-α, IFN-γ. and IL-17 in CD4+ helper T cells. Finally, as shown at block 117, MSCs may prevent and/or inhibit the delivery of cysteine from DCs 116 to T cells 114.

Effects of Exogenously Administered and/or Injected MSCs

As described above herein, the exogenous administration and/or injection of MSCs has the potential to treat various malignant diseases, including, but not limited to, different types of cancers. First, as a practical matter, MSCs generally do not express costimulatory molecules, which are cell surface molecules that can either amplify or inhibit activating signals provided by the TCR to T cells, thereby influencing T cell differentiation. Accordingly, MSCs have relatively low immunogenicity, meaning that there is little or no need to administer immunosuppressive agents in conjunction with, or after, exogenous administration of MSCs. Second, as mentioned previously herein, exogenously administered and/or injected MSCs can use one or more of the same molecular and cellular mechanisms as CA-MSCs to suppress and/or inhibit anti-tumor immunity.

In at least one embodiment, one or more types of MSCs comprised in one or more MSC Compositions and/or one or more MSC Formulations can be genetically modified to express various biological factors, including interleukins (e.g., IL-12). Such modified MSCs may exhibit stronger and/or more sustained expressions and/or secretions of IL-12 and IFN-γ. Accordingly, exogenous administration and/or injection of these genetically modified MSCs may result in stronger anti-tumor T cell responses.

As a further non-limiting example, in murine metastatic models of lung cancer, intravenously injected BM-MSCs significantly augmented lung cancer metastasis by downregulating the anti-tumor immune response. Thus, in at least one embodiment, one or more types of MSCs comprised in one or more MSC Compositions and/or one or more MSC Formulations suppress production of TNF-α in DCs and macrophages, as well as inducing polarization of TNF-α-producing CD4+ Tₕ1 cells and IL-17-producing Tₕ17 cells in IL producing Tregs. Accordingly, serum levels of various anti-tumorigenic cytokines (e.g., TNF-α and IL-17) were decreased, and the serum concentration of immunosuppressive IL-10 was increased in MSC-treated animals with tumors. In at least an additional embodiment, one or more types of MSCs comprised in one or more MSC Compositions and/or one or more MSC Formulations suppress cytotoxicity of CTLs and NK cells in metastatic lungs by (1) downregulating the expression of, for instance, FASL and NKG2D, and (2) reducing exocytosis of, for instance, perforins and granzymes. Additionally, pharmacological inhibition of IDO and iNOS activity completely abrogated MSC-driven suppression of anti-tumor immunity in tumor-bearing mice. This suggests that MSC-sourced IDO and NO were mainly responsible for the pro-tumorigenic effects of MSCs, at least in the context of the aforementioned murine metastatic model.

Accordingly, in at least one embodiment of the disclosure, a method for treating a disease (e.g., cancer) in a subject comprises one or more of: determining that the subject is in need of treatment with one or more MSC Compositions and/or one or more MSC Formulations, the one or more MSC Compositions and/or one or more MSC Formulations including BM-MSCs, administering the one or more MSC Compositions and/or one or more MSC Formulations via one or more administration pathways, such as, for instance, intravenous injection. Such administration may result in (1) suppressing the production of TNF-α in DCs and/or macrophages, (2) inducing polarization of TNF-α-producing CD4+ Tₕ1 cells and IL-17-producing Tₕ17 cells in IL-10-producing Tregs, (3) decreasing serum concentration and/or levels of TNF-α and/or IL-17, (4) increasing serum concentration and/or levels of IL-10, and/or (5) suppressed cytotoxicity of CTLs and NK cells.

In at least a further embodiment, the aforementioned suppression of cytotoxicity of CTLs and NK cells may be achieved by, for instance, (1) downregulating the expression of one or more biological compounds selected from the group consisting of: FASL, NKG2D, and combinations thereof, and/or (2) reducing exocytosis of one or more biological compounds selected from the group consisting of: one or more perforins, one or more granzymes, and combinations thereof.

In at least an additional embodiment, the method further comprises inhibiting IDO and/or iNOS activity, leading to a reduction in measurable levels of IDO and/or NO.

Within the tumor microenvironment, MSCs are constantly exposed to growth factors and/or cytokines released by tumor-infiltrating immune cells, endothelial cells, and/or tumor cells. Although MSCs have some pro-tumorigenic potential, there is no indication that MSCs are natively or constitutively immunosuppressive cells. Rather, MSCs act as a double-edged sword with respect to anti-tumor immunity. As plastic cells, MSCs may adopt the phenotype and/or function of various immune system cells, depending on the influence of one or more biological factors to which they are exposed. Thus, MSCs may obtain either pro-inflammatory (e.g., MSC1) or anti-inflammatory (e.g., MSC2) phenotypes, depending on the local tissue concentration of various inflammatory cytokines, such as, for instance, TNF-α and IFN-γ.

Specifically, when MSCs engraft in a specific tissue that has low levels of TNF-α and IFN-γ, they obtain a pro-inflammatory MSC1 phenotype and secrete a large number of inflammatory factors (e.g., reactive oxygen species (ROS), IL-1(3, interferon alpha and beta (IFN-α, IFN-β), TNF-α, and IFN-γ). These factors can enhance the phagocytic properties of neutrophils and macrophages, as well as enhancing the cytotoxicity of CTLs and NK cells. By contrast, in at least one embodiment, when MSCs are exposed to high levels of inflammatory cytokines (e.g., TNF-α, IFN-γ), they acquire an immunosuppressive MSC2 phenotype characterized by, for instance, the increased production of anti-inflammatory factors (e.g., TGF-β, IL-10, PGE2, NO, IDO, IL-1Ra). These anti-inflammatory factors can suppress the effector function of inflammatory immune cells and attenuate on-going inflammation. Additionally, TNF-α and IFN-γ-primed MSC2 express and secrete PD-L1 and PD-L2, which suppress the proliferation of TNF-α and IFN-γ-producing T cells and promote the generation and/or expansion of immunosuppressive Tregs.

Indeed, the effects of exogenously administered and/or injected MSCs on anti-tumor immunity and/or tumor progression can depend on the time of inoculation, at least in tumor-bearing animal models. In at least one embodiment, one or more types of MSCs comprised in one or more MSC Compositions and/or one or more MSC Formulations are administered during the initial phase of melanoma growth, thereby exerting a tumor-suppressive effect. By contrast, MSCs injected during the progressive stage of melanoma development suppressed anti-tumor immunity and enhanced tumor expansion. MSCs intravenously injected twenty-four (24) hours after melanoma induction significantly enhanced the cytotoxicity of CD8+ CTLs and NK cells, increased the production of anti-tumorigenic cytokines (e.g., TNF-α, IFN-γ, IL-17) in tumor-infiltrated CD4+ Tₕ1 and Tₕ17 lymphocytes, and attenuated melanoma growth and progression.

The opposite findings were observed in melanoma-bearing mice in which MSCs were intravenously injected fourteen (14) days after tumor injection. The MSCs induced suppression of anti-tumor immunity, resulting in enhanced tumor growth, weight loss, and decreased survival. Additionally, MSCs significantly reduced the total number of tumor-infiltrated, MHC class II and cluster of differentiation 80 (CD80)-expressing, IL-12-producing DCs and TAMs. The MSCs also attenuated their antigen-presenting properties. Further, the injected MSCs downregulated secretion of perforin and granzyme B-containing vesicles from activated CTLs and NK cells, reducing their tumoricidal potential. Moreover, MSCs injected 14 days after tumor induction induced the generation of an immunosuppressive phenotype in CD4+ T lymphocytes and prevented the trans-differentiation of TGF-β and IL-10-producing Tregs into anti-tumorigenic IFN-γ- and IL-17-producing Tₕ1 and Tₕ17 cells. Since low levels of inflammatory cytokines (e.g., TNF-α, IFN-γ) were measured in plasma samples of tumor-bearing mice 24 hours after tumor induction, and since concentrations of these inflammatory cytokines were increasing during tumor progression, MSCs injected during the initial phase of melanoma development may have engrafted in a “pro-MSC1 tumor microenvironment” and obtained the anti-tumorigenic MSC1 phenotype. By contrast, MSCs that were administered during the progressive stage of melanoma progression were engrafted in a “pro-MSC2 tumor microenvironment” and, consequently, acquired the immunosuppressive and pro-tumorigenic MSC2 phenotype.

Accordingly, in at least one embodiment of the disclosure, a method for treating a disease (e.g., cancer) in a subject comprises exposing one or more types of MSCs (e.g., BM-MSCs) to sufficiently low levels of TNF-α and/or IFN-γ, thereby (1) obtaining MSCs having a pro-inflammatory MSC1 phenotype, (2) obtaining MSCs that secrete TNF-α and/or IFN-γ, (3) obtaining MSCs that do not express and/or secrete PD-L1 and/or PD-L2, and/or (4) obtaining MSCs that do not promote the generation and/or expansion of immunosuppressive Tregs. The method may further comprise generating a pharmaceutically acceptable composition and/or formulation that includes the exposed MSCs and administering the pharmaceutically acceptable composition and/or formulation to the subject.

In at least a further embodiment, the method comprises administering the pharmaceutically acceptable composition and/or formulation during an initial phase of tumor growth. The administering may be achieved using one or more processes, including, but not limited to, intravenous, intraosseous, intraperitoneal, submandibular, and/or one or more other injection processes. The administered pharmaceutically acceptable composition and/or formulation thereby results in at least one of (1) enhancing the cytotoxicity of CD8+ CTLs, (2) enhancing the cytotoxicity of NK cells, (3) increasing the production of one or more cytokines in CD4+ Tₕ1 lymphocytes, (4) increasing the production of the one or more cytokines in CD4+ Tₕ17 lymphocytes. The aforementioned one or more cytokines may be selected from the group consisting of: TNF-α, IFN-γ, IL-17, and combinations thereof.

MSCs as Therapeutic Agents in Cancer Immunotherapies

There are several mechanisms by which MSCs can be used as therapeutic agents in the treatment of various tumors and/or cancers. For instance, MSCs do not express MHC class II molecules; accordingly, in at least one embodiment, one or more types of MSCs comprised in one or more MSC Compositions and/or one or more MSC Formulations are transplanted into MHC mismatched recipients. Additionally, in at least another embodiment, one or more types of MSCs comprised in one or more MSC Compositions and/or one or more MSC Formulations express various chemokine receptors (e.g., C-X-C chemokine receptor type 4 (CXCR4), C-X3-C chemokine receptor 1 (CX3CR1), C-X-C chemokine receptor type 6 (CXCR6), C-X-C chemokine receptor type 2 (CXCR2), C-C chemokine receptor type 1 (CCR1), C-C chemokine receptor type 7 (CCR7)). In at least a further embodiment, one or more C-X-C motif ligands (CXCL) are also administered, such as, for instance, CXCL2, CXCL3, CXCL13, CXCL14, CXCL16, and/or CXCL17. In at least another embodiment, a method comprises administering one or more types of MSCs that express one or more of the aforementioned chemokine receptors to a subject, thereby resulting in the one or more type of MSCs migrating to tumor tissue in the subject.

A non-limiting list of potential MSC-based and/or MSC-mediated therapies is listed below.

MSC-based therapies for cancer treatments.

| Therapy | | Route of | Specific Disease and/or |
|---|---|---|---|
| No. | Type of MSCs | Injection/Administration | Tumor Type |
| 1 | IFN-β-expressing | intraperitoneal | ovarian cancer |
| | MSCs | | |
| 2 | Adipose tissue- | submandibular | radiation-induced xerostomia |
| | derived MSCs (“AT- | | in previous head and neck |
| | MSCs”) | | cancer patients |
| 3 | MSCs plus umbilical | intraosseous | hematological malignancies |
| | cord hematopoietic | | |
| | stem cells (“UC- | | |
| | HSCs”) | | |
| 4 | BM-MSCs infected | intravenous | metastatic and refractory |
| | with an oncolytic | | tumors |
| | adenovirus, | | |
| | ICOVIR-5 (i.e., | | |
| | Celyvir) | | |
| 5 | BM-MSCs | intravenous | Acute respiratory distress |
| | | | syndrome (ARDS) in patients |
| | | | with malignancies |
| 6 | MSC infected with | intraperitoneal | recurrent ovarian, primary |
| | oncolytic measles | | peritoneal, and fallopian tube |
| | virus encoding | | cancer |
| | thyroidal sodium | | |
| | iodide symporter | | |
| | (“MV-NIS”) | | |
| 7 | TRAIL-expressing | intravenous | metastatic non-small cell lung |
| | MSCs plus | | cancer (NSCLC) |
| | cisplatin/pemetrexed | | |
| 8 | BM-MSCs infected | intravenous | Diffuse intrinsic pontine |
| | with an oncolytic | | glioma (DIPG) |
| | adenovirus, | | medulloblastoma |
| | ICOVIR-5 (i.e., | | |
| | Celyvir) | | |

As shown in the above table, and in at least one embodiment, MSCs can be exogenously administered and/or injected either alone or in conjunction with other cells and/or compounds (e.g., UC-HSCs). Further, in at least an additional embodiment, the one or more MSC Compositions and/or one or more MSC Formulations comprise MSCs that are purposefully infected with one or more oncolytic viruses, such as ICOVIR-5. Generally, oncolytic viruses (also referred to as “OV” or “OVs”) are viruses, either genetically engineered or naturally occurring, that selectively replicate in cancer cells, harming and/or killing only the cancer cells and not any surrounding healthy cells. OVs are usually administered using a delivery vehicle (e.g., MSCs) since direct administration of OVs generally results in the immune system clearing the OVs before they reach the tumor site. Various OVs exist, including, for instance, oncolytic adenovirus (also referred to as “OAV” or “OAVs”), oncolytic herpes simplex virus (HSV) (also referred to as “OHSV” or “OHSVs”), and oncolytic measles virus (also referred to as “OMV” or “OMVs”).

In at least one embodiment, MSCs are used as a delivery vehicle for OAVs (also referred to as “MSC-OAV” or “MSC-OAVs”), which can exhibit anti-tumor properties. In at least an additional embodiment, such MSCs can inhibit IFN-γ production by activated T cells, in addition to promoting uptake of OAVs in the tumor cells. Exogenously administered and/or injected MSC-OAVs may also effectively home to tumor sites (e.g., hepatocellular carcinoma tumors) and inhibit tumor growth and/or development. In at least a further embodiment, a method comprises exogenous administration and/or injection of one or more MSC Compositions and/or one or more MSC Formulations that comprise MSC-OAVs to treat a subject with one or more tumors and/or cancers, non-limiting examples of which include hepatocellular carcinoma (HCC), lung cancer, breast cancer, pancreatic cancer, neuroblastoma, colorectal cancer, and prostate cancer.

In at least one embodiment, the specific OAV is ICOVIR-5, a virus that may exhibit increased replication in tumor cells when compared with other OAVs. Generally, ICOVIR-5 acts by controlling expression of the E1a-Δ24 gene under an E2F Transcription Factor 1 (E2F1) promoter that is insulated with DM-1, the myotonic dystrophy locus insulator. ICOVIR-5 further contains the so-called Kozak consensus sequence (also referred to as the “Kozak consensus” or “Kozak sequence”) immediately before the first codon of the Ela gene. The Kozak sequence is a nucleic acid sequence that functions as a protein translation initiation site that optimizes translation of mRNA by ribosomes. This may result in increased oncolytic and anti-tumor activity.

In at least one embodiment, MSCs can also be used to deliver OHSVs. Such MSCs (also referred to as “MSC-OHSV” or “MSC-OHSVs”) can result in lysis of cancer cells (e.g., glioblastoma cells). Further, MSC-OHVs may, at least in animal models, stimulate apoptosis of cancer cells, leading to reduced tumor growth and reduced and/or absent metastases. In at least an additional embodiment, a specific OHSV used is HF10, a mutant form of HSV-1. In at least a further embodiment, a method comprises exogenous administration and/or injection of one or more MSC Compositions and/or one or more MSC Formulations comprising MSC-OHSVs to treat a subject with one or more tumors and/or cancers, non-limiting examples of which include pancreatic cancer, melanomas, and ovarian cancer. The method may further comprise administration in combination with other agents (e.g., the tyrosine kinase inhibitor erlotinib). Without wishing to be bound by theory, such MSCs may result in high levels of cytotoxicity towards specific tumor cells and/or cell lines (e.g., human pancreatic cell lines) when compared to controls. Moreover, the combination of MSCs with HF10 and erlotinib may further result in more persistent viral presence and/or replication in tumor sites, leading to more prolonged uptake of the virus by tumor cells. Additional non-limiting examples of tumors and/or cancers that may be inhibited by exogenous administration and/or injection of MSC-OHSVs include melanomas and ovarian cancer.

In at least one embodiment, MSCs are used to encapsulate and/or deliver OMVs (also referred to as “MSC-OMV” or “MSC-OMVs”). In at least one example, exogenously administered and/or injected MSC-OMVs can home to specific tumors (e.g., peritoneal tumors) and cause viral infection in those tumors. At least in animal models, such infections may occur regardless of whether the animals were previously immunized against the measles virus. Moreover, administration of MSC-OMVs may provide anti-tumor benefits that are not provided by either (1) administration of MSCs alone, or (2) administration of OMVs alone. Such anti-tumor benefits may be due to, for instance, increased induction of apoptosis. Additional non-limiting examples of tumors and/or cancers that may be inhibited by exogenous administration and/or injection of MSC-OMVs include ovarian cancer, HCC, and acute lymphocytic leukemia (ALL). Accordingly, in at least an additional embodiment, a method comprises exogenous administration and/or injection of one or more MSC Compositions and/or one or more MSC Formulations comprising MSC-OMVs to treat a subject with one or more tumors and/or cancers, non-limiting examples of which include ovarian cancer, HCC, and ALL.

Further, in at least one embodiment of the disclosure, a method for treating a disease (e.g., one or more of the cancers listed in Table 1) comprises injecting and/or administering one or more MSC Compositions and/or one or more MSC Formulations comprising one or more of the MSC types listed in Table 1. Such MSC types may include, for instance, at least one of: IFN-β-expressing MSCs, AT-MSCs, UC-HSCs, MSC-OAVs, MSC-HSVs, MSC-OMVs, BM-MSCs, MV-NIS, and TRAIL-expressing MSCs. The one or more type of MSCs may be administered with one or more additional compounds and/or adjuvants, including, for instance, pharmaceutical compounds (e.g., cisplatin, pemetrexed). The aforementioned injecting and/or administering may be performed via any of the routes listed in Table 1, including, for example, intraperitoneal, submandibular, intraosseous, intravenous, and combinations thereof.

Further MSC-Mediated Therapies

In at least one embodiment, MSCs can be used as vehicles for delivering bi-specific T-cell engaging antibodies. Without wishing to be bound by theory, MSCs can be used as such vehicles due to their low immunogenicity and tumor-homing properties. The aforementioned antibodies are protein engagers that simultaneously bind to tumor antigens and the appropriate ligand on one or more T lymphocytes, thereby enabling specific T cell-mediated elimination of tumor cells. Accordingly, in at least an additional embodiment, a method comprises exogenous administration and/or injection of one or more MSC Compositions and/or one or more MSC Formulations comprising MSCs that encapsulate one or more bi-specific T-cell engaging antibodies.

In at least one embodiment, MSCs are used to encapsulate and/or deliver glypican 3 (GPC3), a protein that regulates the proliferation of hepatocellular carcinoma cells. Hedgehog (“Hh”) pathway signaling can regulate one or more aspects of hepatocellular carcinoma tumorigenesis, and GPC3 can regulate Hh signaling. In at least one example, the delivered GPC3 can inhibit expression of one or more genes in the Hh pathway. Such inhibitory effects can be themselves reduced by heparin, a glycosaminoglycan that is a competitor for GPC3 binding.

In at least one embodiment, MSCs can be genetically modified with one or more viral vectors encoding a GPC3/cluster of differentiation 3 (CD3) bi-specific T cell engager. In at least an additional embodiment, MSCs that express the GPC3-specific single chain variable fragment (“scFv”) and the CD3-specific scFv (“MSCGPC3-CD3” or “MSCsGPC3-CD3”) can direct GPC3-specific CD4+ T helper cells and CD8+ CTLs towards GPC3-expressing hepatocellular carcinoma cells. In at least a further embodiment, co-cultures of GPC3+ tumor cells, MSCsGPC3-CD3s, and T lymphocytes can lead to an increased production of IFN-γ in GPC3-specific CD4+ T cells, as well as an enhanced activation and expansion of GPC3-specific CTLs. These effects resulted in CTL-dependent killing of GPC3-expressing malignant cells. Similar results can occur in vivo. In at least another embodiment, in MSCsGPC3-CD3-treated tumor-bearing mice, there may be an increased activation of GPC3-specific T cells and a concomitant significant reduction in hepatocellular carcinoma growth. Thus, MSCsGPC3-CD3 have the potential for treating, either alone or in combination with other compounds and/or treatments, hepatocellular carcinoma.

Accordingly, in at least an additional embodiment, a method comprises exogenous administration and/or injection of one or more MSC Compositions and/or one or more MSC Formulations comprising MSCs that encapsulate GPC3 to treat a subject with one or more tumors and/or cancers, non-limiting examples of which include hepatocellular carcinomas.

In at least one embodiment, administration of one or more MSC Compositions and/or one or more MSC Formulations is combined with one or more low doses of ultraviolet (UV) radiation and/or X-ray irradiation, thereby generating the anti-tumorigenic MSC1 phenotype in MSCs. In at least an additional embodiment, such radiation is used for MSC priming, and irradiated MSCs can be used as an immunotherapy in combination with other radiation-based therapies. Irradiated BM-MSC1 cells can secrete large amounts of TNF-α and/or IFN-γ which result in several effects, including, for instance, (1) inhibiting the proliferation of tumor cells by deregulating Wnt and TGF-β/Smad signaling, and (2) inducing apoptosis of tumor cells by, for instance, blocking their cell cycle in the G1 phase. Further, irradiation of MSCs can (1) induce cleavage of caspase-3, a protein that, along with other caspase proteins, plays a role in apoptosis, (2) attenuate the phosphorylation of phosphatidylinositol 3-kinase (PI3K)/protein kinase B (PKB, also referred to as AKT), and (3) attenuate the phosphorylation of extracellular signal-regulated kinase. Accordingly, in at least an additional embodiment, a method comprises exogenous administration and/or injection of one or more MSC Compositions and/or one or more MSC Formulations comprising MSCs in combination with one or more low doses of UV radiation and/or X-ray irradiation. In at least another embodiment, one or more types of MSCs are primed with such irradiation before administration and/or injection into a subject.

In at least one embodiment, MSC-sourced TNF-α can induce necrosis of tumor cells and enhance the expression of specific selectins (e.g., E-family selections, P-family selectins) on tumor endothelial cells, enabling an influx of immune cells. In at least an additional embodiment, MSC-sourced IFN-γ can also induce the generation of the anti-tumorigenic M1 phenotype in TAMs and can enhance the cytotoxicity of tumor-infiltrated CTLs and/or NK cells. Upon activation by MSC-derived IFN-γ, CD8+ CTLs and/or NK cells can upregulate the expression of, for instance, FASL and TRAIL, and increase the release of perforins and/or granzymes that induce apoptosis of tumor cells. up-regulate expression of FASL and TRAIL and increase release of perforin and granzymes that induce apoptosis of tumor cells. IFN-γ-primed M1 macrophages can either (1) phagocyte apoptotic tumor cells, or (2) secrete ROS, NO, and TNF-α, which have direct cytotoxic effects on malignant cells. Accordingly, in at least another embodiment, a method comprises exogenous administration and/or injection of one or more MSC Compositions and/or one or more MSC Formulations comprising (1) MSC-sourced TNF-α, and/or (2) MSC-sourced IFN-γ. In at least another embodiment, the method comprises exogenous administration of (1) MSC-sourced TNF-a, and/or (2) MSC-sourced IFN-γ.

Since MSCs may have a high affinity for tumor tissue, in at least one embodiment, one or more MSC Compositions and/or one or more MSC Formulations comprise one or more types of MSCs that are used as targeted delivery vehicles and/or agents of one or more treatment compounds (e.g., prodrugs, anti-cancer drugs, including, for instance, one or more chemotherapeutic drugs, and the like). Non-limiting examples of such compounds include gemcitabine (GCB), paclitaxel (PTX), doxorubicin (DOX), 5-fluorocytosine (5-FC), ganciclovir (GCV), and one or more immune-activating cytokines. In at least an additional embodiment, a method comprises exogenous administration and/or injection of one or more types of MSCs loaded with the anti-cancer drug PTX, which can result in reduced numbers of lung metastases, at least in melanoma-bearing animals. MSCs loaded with PTX may also exhibit anti-tumor properties against other types of cancer as well (e.g., ovarian cancer). Moreover, MSCs may be able to uptake and secrete chemotherapeutic agents (e.g., PTX, DOX, GCB). In leukemia models, MSCs secreting PTX can reduce the ability of leukemia cells to adhere to the microvascular endothelium (MEC) by negatively regulating, for instance, MEC expression of intercellular adhesion molecule-1 (ICAM-1) and vascular cell adhesion molecule-1 (VCAM-1).

In at least one embodiment, one or more MSC Compositions and/or one or more MSC Formulations comprise one or more types of MSCs that are used as targeted delivery vehicles and/or agents for DOX. MSCs primed with DOX may likewise induce anti-tumor effects against various tumor cells (e.g., breast cancer cells, anaplastic thyroid cancer cells). In at least an additional embodiment, DOX is loaded into one or more engineered particles (e.g., nanoparticles) coated with MSC or MSC-like membranes. Without wishing to be bound by theory, such coated particles can distribute DOX more effectively, and with fewer side effects, than general systemic administration.

In at least one embodiment, one or more MSC Compositions and/or one or more MSC Formulations comprise one or more types of MSCs that are used as targeted delivery vehicles and/or agents for one or more prodrugs. In at least an additional embodiment, one or more types of MSCs are engineered to express particular enzymes (e.g., HSV-thymidine kinase (also referred to as “HSV-TK”), cytosine deaminase) that can convert various prodrugs (e.g., 5-FC, GCV) into their active cytotoxic forms. For instance, MSCs modified to express HSV-TK can phosphorylate GCV into its cytotoxic metabolites, thereby resulting in anti-tumor effects. In at least a further embodiment, one or more types of MSCs are transduced with one or more vectors (e.g., lentivectors) expressing HSV-TK. In at least another embodiment, a method comprises exogenous administration and/or injection of such modified MSCs, either alone or in combination with subsequent administration of GCV. Without wishing to be bound by theory, such combination treatment can result in anti-tumor effects, at least in murine cancer models. Such anti-tumor effects may result from, for instance, activating NK cell and/or CTL anti-tumor functions. In at least another embodiment, one or more types of MSCs that are modified to express HSV-TK can be synergistically combined with other agents (e.g., valproic acid (VPA)). Such combination therapy can cause induction of apoptosis in glioma cells; this effect may occur via, for instance, caspase activation. In at least another embodiment, one or more types of MSCs are genetically engineered to express both HSV-TK and TRAIL, which can reduce tumor nodule frequencies, at least in murine lung cancer models, when compared to treatment with controls. Such effects can be sustained and/or increased via routine, serial injections. In at least another embodiment, at least with respect to pancreatic cancer models, one or more types of MSCs engineered to express HSV-TK can home into primary pancreatic tumor stroma and induce C-C motif chemokine ligand 5 (CCLS) promoter activation. Since CCLS expresses a chemokine that functions as a chemoattractant for various immune system cells (e.g., memory helper T cells), administration MSCs engineered to express HSV-TK can result in anti-tumor effects, including, for example, inhibition of primary pancreatic tumor growth and/or occurrence of metastases.

In at least one embodiment, one or more MSC Compositions and/or one or more MSC Formulations comprise one or more types of MSCs that are engineered to express cytosine deaminase, which can convert 5-FC into the cytotoxic antineoplastic 5-fluorouracil (5-FU). In at least another embodiment, a method comprises exogenous administration and/or injection of BM-MSCs expressing cytosine deaminase. This can result, at least in murine models, in lower tumor masses and weights when the mice are subsequently treated with 5-FC, as compared to treatment with 5-FC alone. In at least a further embodiment, such MSCs are administered in combination with one or more other agents (e.g., temozolomide (TMZ), an alkylating agent used to treat glioblastoma multiforme). Indeed, MSCs expressing cytosine deaminase may synergistically interact with TMZ to hinder glioma cell proliferation by, for instance, inducing cell cycle arrest and/or DNA breakage. In at least another embodiment, other combination therapies are used, including, for example, administering MSCs expressing cytosine deaminase with lysomustine, a nitrosourea derivative of lysine, followed by administration of 5-FC. Such a treatment protocol, at least in murine models, can result in a reduction of late-stage Lewis lung carcinoma (LLC) tumor volume and/or tumor growth.

It should be appreciated that using one or more types of MSCs for targeted drug and/or prodrug delivery, according to embodiments described herein, can have several advantages to other drug administration protocols and/or routes. MSCs can be administered at the site of both primary and metastatic tumors, and their targeted nature minimizes side effects that are common with other cancer treatments (e.g., systemic application of chemotherapeutic drugs). In other words, drug loaded MSCs can release chemotherapeutic drugs directly at the tumor site without affecting neighboring tissues. This may result in an increased half-life for the chemotherapeutic compounds, as well as more significant anti-tumor effects.

In at least one embodiment, one or more MSC Compositions and/or one or more MSC Formulations comprise one or more types of MSCs that are used as targeted delivery vehicles and/or agents for one or more cytokines, including, for instance, IL-2, IL-12, IL-21, and TRAIL. In at least another embodiment, the one or more types of MSCs produce IL-2. This can assist CD8+ cells in anti-tumorigenic responses, at least in murine models of melanoma and glioma. In at least an additional embodiment, exogenous administration and/or injection of MSCs producing IL-12 can produce anti-tumor effects in murine models of various cancers (e.g., melanoma, cervical cancer, renal cell carcinoma (RCC), breast cancer, and glioma. IL-12-producing MSCs may have several effects, including, for example, activating NK cells and increasing IFN-γ secretion. MSCs producing IL-21 may also promote IFN-γ secretion and NK cell cytotoxicity.

In at least one embodiment, one or more MSC Compositions and/or one or more MSC Formulations comprise one or more types of MSCs genetically engineered to express TRAIL. Such MSCs, as discussed above herein, are a potentially interesting immunotherapy treatment option since TRAIL uniquely targets cancer cells without harming nearby, non-cancerous cells. As mentioned above herein, the presence of TRAIL-specific receptors also referred to as death receptors, is much higher in cancer cells than non-cancer cells. Accordingly, in at least an additional embodiment, TRAIL can be used in various immunotherapies, including, but not limited to, the therapies discussed herein, in either a full-length and membrane-bound form or a modified form generally referred to as “soluble TRAIL” or “sTRAIL.” MSCs expressing TRAIL can migrate to tumor sites, including lung tumors, where they can induce apoptosis. Such apoptotic effects may also occur in other cancers, including, for example, pancreatic cancers, mesothelioma, renal cancer, breast cancers, neuroblastomas, and non-small cell lung cancers. In at least a further embodiment, MSCs expressing TRAIL may be able to target certain cancer stem cells (e.g., cluster of differentiation 133 (CD133)-positive cancer stem cells), at least in the context of non-small cell lung cancer, resulting in reduction of their proliferation and/or promotion of apoptosis. Such effects may be due to, for instance, modification of the expression of various factors (e.g., nuclear factor-1BI (NF-iB1), BAG cochaperone 3 (BAG3), myeloid cell leukemia-1 (MCLI), DNA damage-inducible alpha (GADD45A), and harakiri (HRK)). In at least another embodiment, MSCs expressing TRAIL can be administered either alone or in combination with one or more other agents, including small-molecule drugs. Administration of both (1) MSCs expressing TRAIL and (2) small-molecule drugs can result in increased tumor sensitivity to TRAIL.

In at least one embodiment, one or more MSC Compositions and/or one or more MSC Formulations comprise one or more types of MSCs that are used as targeted delivery vehicles and/or agents for one or more interferons, including, for example, IFN-α, IFN-β, and IFN-γ. IFN-β-producing MSCs can downregulate various factors, including, for instance, STAT3, Src, Akt, cMyc, MMP2, VEGF, and IL-6. Additionally, in at least a further embodiment, exogenous administration and/or injection of MSCs expressing IFN-α can inhibit tumor growth, including lung cancer metastases, at least in murine models. Such inhibitory effects may result from, for example, activation of NK cells and CD8+ T cells. Further, MSCs expressing IFN-γ can activate the TRAIL pathway, which is responsible for inducing apoptosis. MSCs expressing IFN-γ may also upregulate caspase-3 activation, leading to apoptosis. In vitro, MSCs expressing IFN-γ can polarize macrophages to the M1 phenotype, as well as inducing cell cycle arrest of tumor cells in the G1 phase.

In at least one embodiment, one or more MSC Compositions and/or one or more MSC Formulations comprise one or more types of MSC-derived exosomes (“MSC-Exo” or “MSC-Exos”). In at least a further embodiment, a method comprises administration of such MSC-Exos. In at least an additional embodiment, the MSC-Exos contain one or more MSC-sourced anti-tumorigenic microRNAs (miRNAs). Generally, exosomes are a subset of extracellular vesicles (“EV” or “EVs”), which are membrane-bound vesicles that can be released extracellularly. Such vesicles can contain various biological compounds, including, for example, proteins, lipids, nucleic acids, metabolites, growth factors, and cytokines. EVs may play a role as intercellular communication regulators in various biological processes.

MSCs can, given their plastic nature, either encourage or suppress tumorigenesis via MSC-Exos. This can occur via, for instance, paracrine signaling. As with MSCs themselves, MSC-derived Exos can exert anti-tumorigenic effects by, for instance, mimicking their parental cells. Further, MSC-Exos are EVs which, due to their nanosized dimensions and lipid envelope, can bypass biological barriers and deliver their cargo directly into the target cells. As a result, in at least a further embodiment, MSC-Exos can be genetically engineered to deliver a variety of anti-tumorigenic compounds, such as MSC-sourced biological molecules (e.g., anti-tumorigenic miRNAs, messenger RNAs (mRNAs), enzymes, cytokines, chemokines, growth factors, immunomodulatory factors) directly into tumor cells. In at least another embodiment, additional biological compounds f (e.g., small-molecule drugs, proteins) are also carried by MSC-Exos. Delivery of anti-tumorigenic compounds and/or molecules directly into a tumor could result in the alteration of tumor cell viability, proliferation rate, and/or invasive characteristics.

In at least one embodiment, the MSC-Exos are engineered to deliver one or more immunoregulatory miRNAs and/or one or more immunomodulatory proteins in one or more immune system cells (e.g., M1 macrophages, DCs, CD4+ Tₕ1, CD4+ Tₕ17 cells), thereby enabling their phenotypic conversion into immunosuppressive M2 macrophages, tolerogenic DCs, and regulatory T cells, respectively. In at least an additional embodiment, MSC-Exos can, via delivery of one or more mRNAs and/or miRNAs, activate autophagy, inhibit apoptosis, necrosis, and/or oxidative stress. Such effects can be seen in a variety of cells, including, for example, injured hepatocytes, neurons, retinal cells, and lung, gut, and renal epithelial cells.

In at least one embodiment, the MSC-Exos contain miRNA-16-5p and miRNA-3940-5p. Such MSC-Exo-sourced miRNA-16-5p and miRNA-3940-5p can inhibit the migratory properties and metastatic potential of tumor cells by, for instance, downregulating the expression of Integrin Subunit Alpha 2 (ITGA2) and Integrin Subunit Alpha 6 (ITGA6) on their membranes. MSC-Exos overexpressing miR-16-5p can inhibit proliferation, migration, and/or invasion of tumor cells (e.g., colorectal cancer cells), as well as repressing general tumor growth. Upregulation of miRNA-3940-5p can inhibit invasion of tumor cells as well; additionally, it can suppress tumor metastasis. Since miRNA-3940-5p can bind directly to ITGA6, overexpression of ITGA6 can promote tumor cell invasion and tumor progression via upregulating TGF-β1 signaling.

In at least one embodiment, the MSC-Exos contain miRNA-4461. Such MSC-Exo-delivered miRNA-4461 can suppress the proliferation and/or invasive properties of tumor cells (e.g., colorectal cancer cells) by, for example, reducing expression of COPI coat complex subunit beta 2 (COPB2), which is essential for Golgi budding and vesicular trafficking. miRNA-4461, which may be under-expressed in tumor cells relative to normal cells, can directly target COPB2.

In at least one embodiment, the MSC-Exos contain miRNA-15a. Such MSC-Exos carrying miRNA-15a can inhibit immune escape of tumor cells by, for instance, regulating the expression of homeobox C4 (HOXC4), which binds to the promoter sequence of PD-L1, controlling its synthesis and membrane expression. MSC-derived miRNA-15a can also induce the apoptosis of tumor cells by, for example, inhibiting the activity of histone lysine demethylase 4B (KDM4B), which epigenetically regulates chromatin structure.

In at least one embodiment, the MSC-Exos contain miRNA-100. Such MSC-Exo-delivered miRNA-100 can downregulate the production and/or secretion of VEGF in cancer cells (e.g., breast cancer cells), preventing the generation of new blood vessels in growing tumors. Such downregulation may occur in a dose-dependent manner and may be due to modulation of the mammalian target of rapamycin (mTOR)/hypoxia-inducible factor 1-alpha (HIF-1a) signaling axis.

Thus, in at least one embodiment, one or more types of genetically engineered MSCs that express one or more bi-specific T-cell engaging antibodies (e.g., GPC3-specific scFv, CD3-specific scFv) and/or produce one or more anti-tumorigenic miRNAs (e.g., miRNA-16-5p, miRNA-3940-5p, miRNA-4461, miRNA-15a, miRNA-100) can be used as therapeutic agents in the immunotherapy of malignant diseases (e.g., various types of cancers). Since MSCs can alter their phenotype and/or function in the tumor microenvironment, MSC-mediated treatments can be further tested to address potential safety concerns related to plasticity of MSCs and their possible pro-tumorigenic effects.

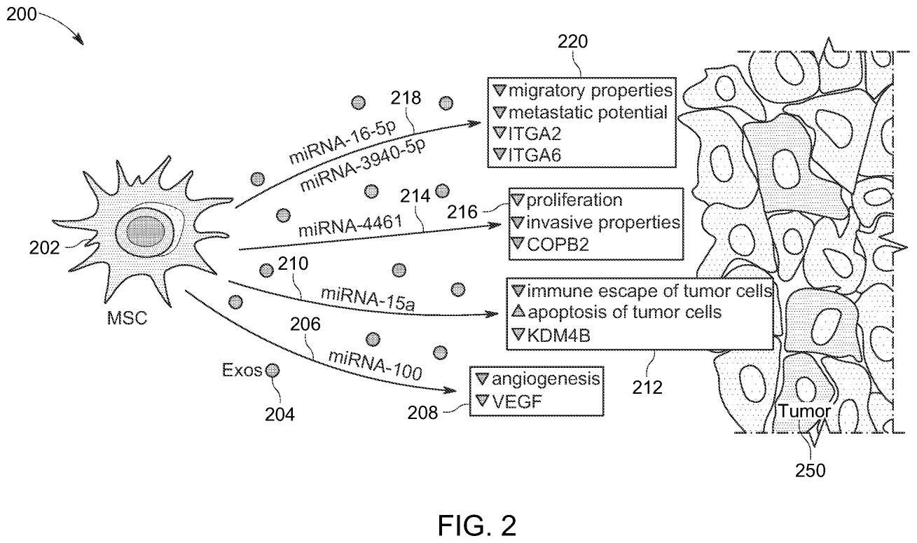

FIG. 2 shows various MSC-based therapies 200 for treating tumors. Exosomes 204 derived from MSCs 202 can contain specific MSC-sourced anti-tumorigenic miRNAs, including, for instance, miRNA-100, miRNA-15a, miRNA-4461, miRNA-16-5p, and miRNA-3940-5p. Administration of one or more of these MSC-sourced anti-tumorigenic miRNAs can have immunotherapeutic effects on tumor 250. Specifically, administration of miRNA-100 can, via pathway 206, result in (1) downregulation of VEGF production and/or (2) decreased angiogenesis, as shown in block 208. Additionally, administration of miRNA-15a can, via pathway 210, result in (1) decreased immune escape of tumor cells, (2) increased apoptosis of tumor cells, and/or (3) inhibiting the activity of histone lysine demethylase 4B (KDM4B), all of which are shown at block 212. Further, administration of miRNA-4461 can, via pathway 214, result in (1) decreased proliferation of tumor cells, (2) reduction in invasive properties of the tumor cells, and/or (3) downregulating expression of COPB2, all of which are shown at block 216. Notably, treating cancer and/or tumors in a subject also comprises prevention of cancer and/or tumors in the subject using the compositions described herein. Finally, administration of miRNA-16-5p and/or miRNA-3940-5p can, via pathway 218, result in (1) inhibiting the migratory properties of tumor cells, (2) inhibiting the metastatic potential of the tumor cells, (3) downregulating expression of ITGA2, and/or (4) downregulating expression of ITGA6, all of which are shown at block 220.

Accordingly, in at least one embodiment of the disclosure, a method for treating a disease (e.g., cancer) comprises administering one or more MSC Compositions and/or one or more MSC Formulations comprising one or more types of MSC that express one or more bi-specific T-cell engaging antibodies, thereby resulting in at least one of (1) directing GPC3-specific CD4+ T helper cells and CD8+ CTLs towards GPC3-expressing tumor cells, (2) increasing production of IFN-γ in GPC3-specific CD4+ T cells, (3) enhancing activation of GPC3-specific CTLs, and (4) enhancing expansion of GPC3-specific CTLs. The administering may be achieved using one or more processes, including, but not limited to, intravenous, intraosseous, intraperitoneal, submandibular, and/or one or more other injection processes. The aforementioned bi-specific T-cell engaging antibodies may include, for instance, GPC3-specific scFv and/or CD3-specific scFv.

In at least an additional embodiment, the method comprises exposing one or more MSCs (e.g., BM-MSCs) to one or more types of radiation, thereby generating an MSC1 phenotype in the one or more MSCs, and administering the one or more irradiated MSCs (e.g., in the context of one or more MSC Compositions and/or one or more MSC Formulations). The one or more types of radiation may include, for instance, UV and/or X-rays. In at least a further embodiment, the irradiated one or more MSCs may secrete TNF-α and/or IFN-γ. In at least an additional embodiment, the method comprises administering MSC-sourced and/or MSC-derived TNF-α and/or IFN-γ, thereby resulting in at least one of (1) deregulating Wnt signaling, (2) deregulating TGF-β/Smad signaling, (3) blocking the cell cycle of one or more tumor cells in the G1 phase, (4) inducing necrosis of the one or more tumor cells, (5) enhancing expression of one or more selections (e.g., E-family selections, P-family selectins) on the one or more tumor cells, (6) inducing generation of an anti-tumorigenic M1 phenotype in TAMs, (7) activating CD8+ CTLs and/or NK cells to upregulate expression of FASL and/or TRAIL, (8) activating CD8+ CTLs and/or NK cells to increase release of perforins and/or granzymes, (9) priming M1 macrophages to phagocyte apoptotic tumor cells, and (10) priming M1 macrophages to secrete one or more of ROS, NO, and TNF-α. The aforementioned administering may be achieved using one or more processes, including, but not limited to, intravenous, intraosseous, intraperitoneal, submandibular, and/or one or more other injection processes.

In at least a further embodiment, the method comprises administering one or more exosomes derived from one or more MSCs, the one or more exosomes comprising one or more anti-tumorigenic microRNAs. Such microRNAs may be selected from the group consisting of: miRNA-16-5p, miRNA-3940-5p, miRNA-15a, miRNA-100, and combinations thereof. The administering may be achieved using one or more processes, including, but not limited to, intravenous, intraosseous, intraperitoneal, submandibular, and/or one or more other injection processes.

In at least one aspect, administration of MSC-Exo-derived miRNA-16-5p and/or MSC-Exo-derived miRNA-3940-5p (e.g., in the context of one or more MSC Compositions and/or one or more MSC Formulations) results in at least one of (1) downregulating expression of ITGA2 on tumor cell membranes, and (2) downregulating expression of ITGA6 on the tumor cell membranes. In at least an additional aspect, administration of MSC-Exo-derived miRNA-4461 results in reducing expression of COPB2 in one or more tumor cells. In at least a further aspect, administration of MSC-Exos-derived miRNA-15a results in at least one of (1) regulating expression of homeobox C4 (HOXC4), which binds to the promoter sequence of PD-L1, thereby controlling PD-L1 synthesis and membrane expression, (2) inhibiting activity of histone lysine demethylase 4B (KDM4B), thereby inducing apoptosis of the one or more tumor cells. In at least a further aspect, administration of MSC-Exos-derived miRNA-100 results in downregulation of VEGF production in the one or more tumor cells, thereby preventing generation of new blood vessels within, and/or in-between, the one or more tumor cells.

Therefore, based on the foregoing description, the subject invention in its various embodiments may comprise one or more of the following features in any non-mutually-exclusive combination: A method for prevention and treatment of cancers and tumors in a subject, the method comprising administering to the subject an effective amount of a pharmaceutical composition and/or formulation comprising one or more types of mesenchymal stem cells (MSCs); The one or more types of MSCs altering one or more responses of one or more immune cells in the subject; The one or more immune cells being selected from the group consisting of: dendritic cells, macrophages, T cells, natural killer (NK) cells, and combinations thereof; The altering one or more responses of one or more immune cells in the subject comprising enhancing and/or inducing one or more endogenous immune cells in one or more tumors present in the subject; The enhancing and/or inducing comprises at least one of: enhancing cytotoxicity of one or more CD8+ cytotoxic T lymphocytes (CTLs), enhancing cytotoxicity of one or more NK cells, increasing production of one or more cytokines in CD4+ Tₕ1 lymphocytes, and increasing production of one or more cytokines in CD4+ Tₕ17 lymphocytes; The one or more types of MSCs being infected with one or more viruses selected from the group consisting of: oncolytic adenoviruses, oncolytic herpes simplex virus (HSV), oncolytic measles virus, and combinations thereof; The method further comprising administering to the subject one or more doses of radiation; The radiation being ultraviolet (UV) radiation and/or X-ray irradiation; The method further comprising administering to the subject one or more additional agents in combination with the pharmaceutical composition and/or formulation; The one or more additional agents being selected from the group consisting of: gemcitabine (GCB), paclitaxel (PTX), doxorubicin (DOX), 5-fluorocytosine (5-FC), ganciclovir (GCV), an adjuvant, an antigen, an excipient, a vaccine, an allergen, an antibiotic, a gene therapy vector, a kinase inhibitor, a co-stimulatory molecule, a Toll-like receptor (TLR) agonist, a TLR antagonist, a therapeutic agent, a prophylactic agent, a diagnostic agent, an antimicrobial agent, an analgesic, a local anesthetic, an anti-inflammatory agent, an anti-oxidant agent, an immunosuppressant agent, an anti-allergenic agent, an enzyme cofactor, an essential nutrient, a growth factor, and combinations thereof; The administering to the subject an effective amount of a pharmaceutical composition and/or formulation comprising one or more types of MSCs further comprising administering, with the pharmaceutical composition and/or formulation, and a pharmaceutically acceptable carrier for injection.

Further, based on the foregoing description, the subject invention in its various embodiments may also comprise one or more of the following features in any non-mutually-exclusive combination: The administering to the subject an effective amount of a pharmaceutical composition and/or formulation comprising one or more types of MSCs preventing cancer in the subject and/or decreases incidence of cancer in the subject; The administering to the subject an effective amount of a pharmaceutical composition and/or formulation comprising one or more types of MSCs reducing tumor weight and/or tumor volume in the subject; The administering to the subject an effective amount of a pharmaceutical composition and/or formulation comprising one or more types of MSCs further comprising administering the pharmaceutical composition and/or formulation systemically and/or at tumor locations in the subject; The administering to the subject an effective amount of a pharmaceutical composition and/or formulation comprising one or more types of MSCs resulting in at least one of: an increased number of cytotoxic NK cells expressing at least one of: interferon gamma (IFN-γ), Fas ligand (FASL), and granzyme B, an increased number of CD4+ helper T cells, and an increased number of CD8+ cytotoxic T lymphocytes (CTLs); The administering to the subject an effective amount of a pharmaceutical composition and/or formulation comprising one or more types of MSCs resulting in at least one MSC in the one or more types of MSCs having an anti-tumorigenic MSC1 phenotype; The at least one MSC having the anti-tumorigenic MSC1 phenotype secreting tumor necrosis factor-alpha (TNF-α) and/or interferon-gamma (IFN-γ); A method of prevention and treatment of cancers and tumors in a subject, the method comprising administering to the subject an effective amount of a pharmaceutical composition and/or formulation comprising one or more types of mesenchymal stem cell (MSC)-derived exosomes; The one or more types of MSC-derived exosomes altering one or more responses of one or more immune cells in the subject; The one or more types of MSC-derived exosomes being derived ex vivo from one or more types of MSCs; The one or more types of MSC-derived exosomes comprising one or more anti-tumorigenic microRNAs selected from the group consisting of: miRNA-16-5p, miRNA-3940-5p, miRNA-4461, miRNA-15a, miRNA-100, and combinations thereof; The administering to the subject an effective amount of a pharmaceutical composition and/or formulation comprising one or more types of MSC-derived exosomes resulting in an increased concentration of at least one of: IL-1Ra, CCL21, CXCR6, and CXCL14; The administering to the subject an effective amount of a pharmaceutical composition and/or formulation comprising one or more types of MSC-derived exosomes resulting in downregulation of vascular endothelial growth factors (VEGF) production; A pharmaceutical composition administered in any of the aforementioned methods; A pharmaceutical composition comprising one or more types of mesenchymal stem cells (MSCs), and one or more pharmaceutically acceptable excipients; The pharmaceutical composition further comprising one or more agents selected from the group consisting of: an adjuvant, an antigen, an excipient, a vaccine, an allergen, an antibiotic, a gene therapy vector, a kinase inhibitor, a co-stimulatory molecule, a Toll-like receptor (TLR) agonist, a TLR antagonist, a therapeutic agent, a prophylactic agent, a diagnostic agent, an antimicrobial agent, an analgesic, a local anesthetic, an anti-inflammatory agent, an anti-oxidant agent, an immunosuppressant agent, an anti-allergenic agent, an enzyme cofactor, an essential nutrient, a growth factor, and combinations thereof; and the pharmaceutical composition further comprising one or more exosomes generated ex vivo from one or more types of MSCs.

### EXAMPLES

### Example 1: Effect of Exogenous Administration of MSCs in a Murine Melanoma Model

This example describes the exogenous administration of MSCs in a murine model of melanoma to determine whether timing of such administration affected MSC-mediated anti-tumor responses and/or anti-tumor immunity.

Cells

MSCs were isolated from bone marrow of C57BL/6 mice, were purchased from Gibco. The murine melanoma cell line B16F10, which is syngeneic to the C57BL/6 background, was obtained from the American Type Culture Collection (ATCC, USA). Both types of cells were grown in Dulbecco's Modified Eagles Medium (DMEM) supplemented with 10% heat-inactivated fetal bovine serum (FBS), 100 IU/mL penicillin G, and 100 μg/mL streptomycin (Sigma-Aldrich, Munich, Germany). The cells were grown at 35° C. in a 5% CO₂ incubator. MSCs in passage 4 and B16F10 cells in passage 4 were used in the experiments below.

Animals

C57BL/6 mice, eight to ten weeks old, were used. Mice were maintained in animal breeding facilities of the Faculty of Medical Sciences, University of Kragujevac, Serbia. All procedures were performed in accordance with the guidelines for the Principles of Laboratory Animal Care and the Guide for the Care and Use of Laboratory Animals, and all animals received humane care according to the criteria outlined in the Guide for the Care and Use of Laboratory Animals (National Institutes of Health publication 86-23, 1985 revision). All experiments were approved by the Animal Ethical Review Board of the Faculty of Medical Sciences, University of Kragujevac, Serbia. Mice were housed in a temperature-controlled environment with a 12-hour light-dark cycle. All mice were fed with standard laboratory chow and were provided water ad libitum. At least eight mice per group were used in each experiment.

Experimental Design

B16F10 cells (specifically 5×10⁵ cells suspended in 200 μL of phosphate-buffered saline (PBS)) were subcutaneously injected in the left flank of C57BL/6 mice. The mice were then immediately divided into four experimental groups. The first experimental group of mice intravenously received MSCs (specifically 5×10⁵ cells suspended in 200 μL of PBS; B16F10+MSC^{1d}-treated mice) one day after injection of B16F10 cells. The second experimental group of B16F10-treated animals intravenously received MSCs (specifically 5×10⁵ cells suspended in 200 μL of PBS; B16F10+MSC^{14d}-treated mice) 14 days after administration of B16F10 cells. Mice from the third and fourth experimental groups intravenously received 200 μL of PBS at comparable time points (i.e., either one day (B16F10+PBS^{1d}-treated mice) or 14 days after B16F10 administration (B16F10+PBS^{14d}-treated animals)). All animals were sacrificed 28 days after the injection of B16F10 cells.

Measurement of Tumors

Once the tumors were palpable, they were measured daily. Tumor volume was calculated using the following formula: V=4/3π*a/2*b/2*c/2, where a=length, b=width, and c=thickness.

Measurement of Cytokines

To measure cytokines in the plasma samples of tumor-bearing mice, blood samples were collected from the facial vein at days 1, 14, and 28 after injection with the B16F10 cells. Mouse blood was kept in tubes with anticoagulant and then centrifuged for 10 minutes at 2000 g at 4° C. Supernatants were then stored at −20° C. until needed. The concentrations of (1) tumor necrosis factor alpha (TNF-α), (2) interferon gamma (IFN-γ), (3) transforming growth factor beta (TGF-β), and (4) interleukin-10 (IL-10) in mouse plasma samples were measured by using enzyme-linked immunosorbent assay (ELISA) sets (R&D Systems, Minneapolis, Minn., USA), according to the manufacturer's instructions.

Isolation of Leucocytes

Forceps and scissors were used to resect subcutaneous tumors en bloc, including any overlying and surrounding skin. After the removal of surrounding skin, tumors were measured and weighed. The tumors were then minced using scissors until all large sections were processed into 1-2 mm pieces, which were digested in 5 mL of DMEM containing 1 mg/mL collagenase I, 1 mM EDTA, and 2% FBS (all from Sigma-Aldrich, Munich, Germany). After incubation for 2 hours at 37° C., the digested tumor tissue was incubated with 4 mL of trypsin and DNase I (Roche Diagnostics), followed by passing through a 40 μm nylon filter. Single-cell suspensions were then processed for flow cytometry analysis.

Flow Cytometry Analysis

The tumor-infiltrating leucocytes were investigated for different cell surface and intracellular markers using flow cytometry. Briefly, cells were incubated with anti-mouse F4/80, CD4, CD8, CD11c, NK1.1, CD80, I-A, granzyme B, and Fas ligand (FASL) monoclonal antibodies conjugated with fluorescein isothiocyanate (FITC), phycoerythrin (PE), peridinin chlorophyll protein (PerCP), or allophycocyanin (APC) (all from BD Biosciences, San Jose, Calif., USA) following the manufacturer's instructions. Immune cells derived from the tumors were concomitantly stained for the intracellular content of TNF-α, IFN-γ, IL-12, IL-4, and IL-17 by using the fixation/permeabilization kit and anti-mouse monoclonal antibodies conjugated with FITC, PE, PerCP, and APC (BD Biosciences). For intracellular cytokine staining, cells were stimulated with 50 ng/mL PMA and 500 ng/mL ionomycin for 5 hours, and GolgiStop (BD Biosciences) was added. Cells were then fixed in Cytofix/Cytoperm, permeated with 0.1% saponin, and stained with fluorescent antibodies. Flow cytometric analysis was then conducted on a BD Biosciences' FACSCalibur machine and analyzed by using the Flowing Software analysis program.

Statistical Analysis

Statistical data was analyzed using statistical package SPSS, version 21. The normality of the distribution was tested with the Kolmogorov-Smirnov test. Results were then analyzed using the Student's T-test. All data were then expressed as the mean±standard error of the mean (SEM). The difference was considered significant when p<0.05.

Results

Results generally showed that MSCs intravenously injected 24 hours after melanoma induction efficiently suppressed tumor growth and progression. However, MSCs intravenously injected 14 days after melanoma induction promoted tumor growth.

Specifically, tumors become palpable in B16F10+MSC^{1d}-treated mice eight days later compared with other experimental groups. Starting from day 18, average tumor volumes were significantly lower in B16F10+MSC^{1d}-treated mice than in B16F10+PBS^{1d}-treated animals (p<0.05). Further, at day 28, the average volume and weight of tumors removed from B16F10+MSC^{1d}-treated mice were significantly lower than melanomas taken from B16F, 10+PBS^{1d}-treated animals.

By contrast, starting from day 18 (that is, four days after MSC injection), the average tumor volumes in B16F10+MSC^{14d}-treated mice were significantly greater than in B16F10+PBS^{14d}-treated mice. Thus, at day 28, the average volume and weight of tumors removed from B16F10+PBS^{14d}-treated mice were significantly lower than those of melanomas of B16F10+MSC^{14d}-treated mice. Further, While the lowest survival rate was observed in B16F10+MSC^{14d}-treated mice, all of the melanoma-bearing animals that received MSCs 24 h after tumor induction survived till the end of the experiment.

Since, as described above herein, MSCs can adopt pro-inflammatory (i.e., MSC1) or immunosuppressive (i.e., MSC2) phenotypes in response to the inflammatory and immunosuppressive cytokines to which they are exposed, the concentration of inflammatory (TNF-α, IFN-γ) and immunosuppressive cytokines (IL-10, TGF-β) in plasma samples of melanoma-bearing mice at the time of MSC administration were analyzed and compared. The ratios of pro-inflammatory to anti-inflammatory cytokines (TNF-α:IL-10, TNF-α:TGF-β, IFN-γ:IL-10, IFN-γ:TGF-β, IL-12:IL-10, and IL-12:TGF-β) were significantly lower in plasma samples of B16F10+PBS^{1d}-treated mice compared to B16F10+PBS^{14d}-treated animals (p<0.001). This suggests that MSCs, administered one day after the injection of tumor cells, were exposed to a higher concentration of immunosuppressive cytokines, while MSCs transplanted 14 days after tumor induction were exposed to a higher concentration of inflammatory cytokines. Thus, MSCs administered during the initial phase of melanoma growth adopted a pro-inflammatory (MSC1) phenotype, while MSCs administered during the progressive stage of melanoma growth adopted an immunosuppressive (MSC2) phenotype.

Further supporting this conclusion is the fact that MSCs administered 24 hours after tumor induction significantly enhanced NK and T-cell driven antitumor immunity. Specifically, the cellular makeup of tumors obtained from B16F10+PBS^{1d}-treated mice and B16F10+MSC^{1d}-treated mice revealed that MSCs, when injected 24 hours after melanoma induction, significantly increased the total number of tumor-infiltrating cytotoxic NK1.1+NK cells (p<0.05). Additionally, there were significantly higher numbers of IFN-γ-producing (p<0.05) and FASL- and granzyme B-expressing (p<0.05) NK cells in the tumors of B16F10+MSC^{1d}-treated mice. This result indicates that MSCs, when administered 24 hours after melanoma induction, enhanced the cytotoxic and antitumorigenic potential of NK cells in tumor-bearing animals.

Additionally, the tumors of B16F10+MSC¹d-treated mice contained significantly higher numbers of both CD4+ helper T cells (p<0.05) and CD8+CTLs (p<0.05) than in melanomas of B16F10+PBS¹d-treated mice. The phenotype and function of these CD4+ helper T cells and CD8+ CTLs revealed that MSCs, when injected 24 hours after melanoma induction, significantly increased the presence of (1) antitumorigenic and IFN-γ- and TNF-α-producing CD4+ Tₕ1 cells (p<0.05 for IFN-γ and p<0.001 for TNF-α), (2) IL-17-producing CD4+ Tₕ17 cells (p<0.001), and (3) IFN-γ- and TNF-α-producing CD8+ CTLs in melanoma-bearing animals.

Similarly, significantly higher plasma levels of the inflammatory and antitumorigenic cytokines TNF-α (p<0.05) and IFN-γ (p<0.05), and significantly lower plasma levels of immunosuppressive cytokines TGF-β (p<0.05) and IL-10 (p<0.05) were observed in B16F10+MSC^{1d}-treated mice. These results further indicate that, when MSCs are transplanted during the initial phase of melanoma growth, MSCs enhance the anti-tumor immune response in melanoma-bearing animals.

By contrast, MSCs, when transplanted 14 days after melanoma induction, attenuated tumoricidal capacity of NK cells, as evidenced by the lower number of tumor-infiltrating granzyme B-expressing NK1.1+ cells in B16F10+MSC^{14d}-treated mice (p<0.05). Further, MSCs injected 14 days after melanoma induction suppressed the tumoricidal capacity of CD8+CTLs, CD4+ Tₕ1, and CD4+ Tₕ17 lymphocytes. Intracellular staining revealed that MSCs suppressed production of tumoricidal cytokines (e.g., IFN-γ and IL-17) in CD4+ Tₕ1 and Tₕ17 cells (p<0.05 for TNF-α and IL-17) and in CTLs (p<0.05 for IFN-γ and IL-17) of B16F10+MSC^{14d}-treated mice. This may have prevented generation of optimal TNF-α, IFN-γ, and IL-17-driven anti-tumor immune responses. Additionally, a significantly lower number of granzyme B-expressing CD8+CTLs were observed in the tumors of B16F10+MSC^{14d}-treated mice (p<0.05), indicating that MSCs injected 14 days after tumor induction significantly reduced the presence of cytotoxic and pro-apoptotic CD8+CTLs in the tumors of melanoma-bearing animals.

Finally, significantly lower levels of antitumorigenic cytokines TNF-α and IFN-γ (p<0.05) and significantly higher levels of TGF-β and IL-10 (p<0.001) were found in plasma samples of B16F10+MSC^{14d}-treated mice. This result indicated that MSCs, when injected during the progressive stage of melanoma development, attenuated anti-tumor immunity by increasing production of immunosuppressive cytokines in tumor-bearing animals.

### Example 2: Effects of MSC-Derived Exosomes and Exosome-Based Products

This example describes generation and therapeutic effects of MSC-derived exosomes (MSC-Exos) and exosome-based products (e.g., products with biomaterials, growth factors, and/or immunomodulatory cytokines derived from MSC-Exos). One such product is Exosomes Derived Multiple Allogeneic Proteins Paracrine Signaling (Exosomes d-MAPPS).

Sample Acquisition

Exosomes d-MAPPS is an engineered biologic product obtained from placental tissue, previously collected from healthy human donors. Blood samples were provided by the donor prior to, or at the time of, collection and were tested by laboratories certified under the Clinical Laboratory Improvement Amendments (CLIA) and were found negative using United States (U.S.) Food and Drug Administration (FDA) licensed tests for detection of, at minimum, hepatitis B virus, hepatitis C virus, human immunodeficiency virus types 1/2, and Treponema Pallidum. Placental tissue samples were obtained with patient consent as well as institutional ethical approval and kept at 4° C. until processed. Samples were engineered as a sterile product, manufactured under current Good Manufacturing Practices (cGMP) regulations and reviewed by the FDA.

Presence of Cytokines, Chemokines, and Growth Factors

The concentrations of cytokines, chemokines, growth factors and their receptors in Exosomes d-MAPPS samples were determined. Briefly, about fifty milliliters of sample was concentrated to 1.0 ml protein with trichloroacetic acid. The acetone-washed protein pellet was then resolubilized in urea, and proteins were processed with dithiothreitol and iodoacetamide and digested with trypsin. Tryptic peptides were quantified and 10 μg was loaded through pressure cell onto a biphasic column for online two-dimensional high-performance liquid chromatography (HPLC) separation (strong-cation exchange and reversed-phase) and concurrent analysis by nanospray using a hybrid mass spectrometer. Three salt cuts of 50, 100, and 500 mM ammonium acetate were performed per sample run, with each followed by a 120-min organic gradient to separate the peptides.

Resultant peptide fragmentation spectra were compared with proteome database concatenated with common contaminants and reversed sequences to control false discovery rates. Peptide spectrum matches (PSMs) were filtered and assigned matched-ion intensities (MITs) based on observed peptide fragment peaks. PSM MITs were summed on a per-peptide basis, and only those uniquely and specifically matching a particular protein were moved onto subsequent analysis. Briefly, peptide intensity distributions were log-transformed, normalized across biological replicates by LOESS, and standardized by median absolute deviation and mean centering across samples as suggested. Peptides were then filtered to maintain at least two hits in one replicate set, and missing values were imputed using a random distribution of low-level values. Peptide abundance trends for each protein were scaled to a specific, well-sampled reference peptide. Sample-to-sample variation was visualized by PCA, Pearson's correlation and hierarchically clustered using the Ward agglomeration method to generate a heat map of protein abundance trends normalized by z-score.

Results

The concentrations of major MSC-derived immunomodulatory molecules were analyzied, specifically, levels of IDO, IL-1ra, IL-10, IL-4, IL-13, IL-18 binding protein (IL-18 Bpa), TGF131 and Latency associated peptide of TGF131 (LAP (TGFβ1), were measured. IL-1Ra was found in high concentrations (1000 pg/μl); MSC-derived IL-1Ra is a naturally occurring cytokine which acts as an inhibitor of inflammatory cytokine IL-1. When IL-1Ra binds to the IL-1 receptor (IL-1R), binding of IL-1 is blocked and pro-inflammatory signal from IL-1 receptor is stopped. In line with these findings, a high concentration of IL-1Ra indicates strong anti-inflammatory and immunomodulatory potential.

Additionally, the main inflammatory cytokines of innate immunity (e.g. TNF-α, IL-1β, IL-12, IL-18) were not detected. Similarly, Th1 (IFN-γ), Th2 (IL-4, IL-5, IL-10, IL-13) and Th17 (IL-17 and IL-22) cytokines were present in non-detectable concentrations, indicating that neither one of the T cell-dependent inflammatory pathways could be elicited by the sample.

Promoting Migration of CXCR6, CCR7, and CXCR4 Expressing Cells

As described above herein, MSCs have a capacity to home towards the site of injury or inflammation where they, in a juxtacrine and/or paracrine manner, suppress detrimental immune responses and ongoing inflammation. MSCs express chemokine-specific receptors (CXCR4, CX3CR1, CXCR6, CCR1, and CCR7) and are attracted by chemokines (CXCL12, CXCL14, CX3CL1, CXCL16, CCL3, CCL19, and CCL21) released from damaged tissues and inflammatory immune cells. MSCs themselves are also able to produce chemokines which, in autocrine manner, enable migration of MSCs towards the site of injury or inflammation. In line with these observations, high concentrations of MSCs-derived chemokine CXCL16 were found in the sample (1500 pg/μl). Since CXCR6, the ligand for CXCL16, is highly expressed on MSCs and immune cells (e.g., memory/effector T cells, NK cells, NKT cells, and plasma cells), high concentrations of this chemokine strongly indicates that MSC-Exos and exosome-derived products such as Exosomes d-MAPPS can be used as a chemoattractant, enabling migration of CXCR6 expressing cells into inflamed or injured tissues.

Similarly, 6Ckine (CCL21) (ligand for CCR7 receptor) was measured in the sample (500 pg/μl. Bearing in mind that CCL21:CCR7 axis is important for migration of MSCs in wounds, homing of naïve T cells in peripheral lymph nodes and for migration of antigen processing, activated DCs into peripheral lymph nodes and T cell-rich fields within injured lungs, synovia, and eyes, high levels of CCL21 could be used for recruitment of CCR7 expressing MSCs and immune cells for treatment of skin, joint, eye, and/or lung inflammatory diseases. In line with these findings, high concentrations (2000 pg/ml) of platelet factor 4 (PF4), which is involved in tissue regeneration and wound repair, was found in the sample as well.

CXCL14 was also detected in the sample (500 pg/μl). CXCL14 specifically binds to CXCR4 and, in a similar manner as CXCL12, is involved in CXCR4-dependant migration of MSCs into injured or inflamed tissues.

In addition to elevated levels of CXCL16, CCL21, PF4, and CXCL14, GRO-well known MSC-derived chemokine with strong immunosuppressive properties was detected (500 pg/μl). Human MSCs secrete GRO-γ which, accompanied with GRO-α, promote conversion of monocyte derived DCs (MDDCs) towards a myeloid suppressive phenotype, enabling generation of tolerogenic myeloid derived suppressor cells (MDSCs). In line with these findings, presence of GRO in the sample strongly indicates potential for generation of MDSCs and MDSCs-based cell therapy of, e.g., autoimmune and chronic inflammatory diseases.

Inducing Neo-Vascularization in a VEGF-Dependent Manner

Since the generation of new blood vessels and re-vascularization are mainly responsible for MSC-dependent regeneration of ischemic tissues, the presence of angiogenesis-related growth factor receptors in the sample was determined. Results indicate the capacity of MSC-Exos and exosome-derived products to induce neo-angiogenesis-based tissue regeneration. High concentrations of VEGFR1 (20000 pg/μl) were found in the sample. VEGFR1 plays a critical role in the migration of MSCs and MSC-based neo-angiogenesis. VEGFR1 also binds VEGF and is expressed by multiple bone marrow-derived cell types, including endothelial progenitor cells and MSCs. BM-derived endothelial progenitor cells and MSCs are mobilized into peripheral blood and recruited to the sites of ischemia in a VEGFR1-dependent manner, where they participate in tissue repair and revascularization. Based on these results, MSC-Exos and exosome-derived products (e.g., Exosomes d-MAPPS) can modulate generation and maturation of BM-derived cells. In line with these observations, high concentrations of granulocyte-macrophage colony-stimulating factor receptor (GM-CSFR) were also noticed in the sample (20000 pg/μl). Since signaling from GM-CSFR can promote a variety of cellular functions, including protection from apoptosis, progression through the cell cycle, early commitment to myelopoiesis, differentiation/maturation of committed progenitors, and multiple activation and motility functions in mature immune cells, MSC-Exos and exosome-derived products (e.g., Exosomes d-MAPPS) can be used for controlled differentiation of BM-derived, GM-CSFR expressing cells.

### Example 3: Treatment of COPD Patients with MSC-Derived Exosome-Based Products

This example describes therapeutic effects of MSC-derived exosome-based products, one of which is Exosomes d-MAPPS, in COPD patients.

Sample Acquisition

Sterile Exosomes d-MAPPS is an engineered biological product obtained from placenta MSCs (PL-MSCs) previously collected from healthy human donors. PL-MSC samples were obtained with patient consent as well as institutional ethical approval and kept at 4° C. until processed. All donors prior to, or at the time of, collection were tested by laboratories certified under the Clinical Laboratory Improvement Amendments (CLIA) and were found negative using United States (U.S.) Food and Drug Administration (FDA) licensed tests for the detection of, at minimum, hepatitis B virus, hepatitis C virus, human immunodeficiency virus types 1/2, and Treponema pallidum.

Exosomes d-MAPPS was engineered as a sterile product and manufactured under current Good Manufacturing Practices (cGMP) regulated and reviewed by the FDA. Briefly, PL-MSCs were grown in complete MSC Dulbecco's Modified Eagle's Medium (DMEM). Low passage (<5) PL-MSCs were grown to 60%-80% confluence in multiflasks before isolation. Fresh PL-MSC media were layered and collected after 48 to 72 hours (conditioned medium). Exosomes (Exos) were isolated by the ultracentrifugation protocol (100,000 g at 4° C. for 70 min). The isolation of Exos was performed by positive selection using the μMACS™ Separator (Miltenyi Biotec, Bergisch Gladbach, Germany) and the Exosome Isolation Kit Pan, human (Miltenyi Biotec, Bergisch Gladbach, Germany) which contained a cocktail of MicroBeads conjugated to the tetraspanin proteins CD9, CD63, and CD81. Briefly, Exos were magnetically labeled and loaded onto a μ column, which was placed in the magnetic field of μMACS™ Separator. The magnetically labeled Exos were retained within the column, while the unlabeled vesicles and cell components run through the column. After removing the column from the magnetic field, the intact Exos were collected by elution. Exos were stored at −70° C. until use.

Animals

For animal studies, eight- to ten-week-old male BALB/c mice were used. Mice were maintained in animal facilities of the Faculty of Medical Sciences, University of Kragujevac, Serbia. All animals received humane care, and all experiments were approved by and conducted in accordance with the Guidelines of the Animal Ethics Committee of the Faculty of Medical Sciences of the University of Kragujevac. Mice were housed in a temperature-controlled environment with a 12-hour light-dark cycle and were administered with standard laboratory chow and water ad libitum.

Experimental Design

Animals were randomly divided into control and experimental groups (n=8 mice per group). Mice from the experimental group underwent whole-body exposure to cigarette smoke (CS) of 5 cigarettes in a CS chamber with 30-minute smoke-free intervals, every day for four weeks. The smoke exposure experimental box, adapted for a group of 8 mice, consisted of a box body and a cover. CS was drawn through an exposure chamber by negative pressure using an extraction pump. Between draws of CS, room air was continuously drawn through the chamber. The smoke-to-air ratio was 1:12 to protect mice from acute smoke toxicity and death.

After four weeks of CS treatment, mice were randomly divided into two groups and received either vehicle or Exosomes d-MAPPS (0.1 mL/intraperitoneally/5 days per week for three weeks). Mice from the control group were exposed to air only and received either vehicle or Exosomes d-MAPP S.

Histopathological Analysis

All mice were sacrificed 8 weeks after initial CS exposure, and the lungs were isolated for histopathological analysis. The isolated lungs were fixed in 10% formalin, embedded in paraffin, and consecutive 4 μm tissue sections were mounted on slides. Sections were stained with hematoxylin and eosin (H&E) and examined under a low-power (100×) light microscope-equipped digital camera (Zeiss Axioskop 40, Jena, Germany).

Blood Gas Analysis

In order to explore whether Exosomes d-MAPPS treatment managed to improve extracellular acid-base status and gas exchange in CS-exposed mice, blood gas parameters (partial pressure of oxygen in arterial blood (PaO₂), partial pressure of carbon dioxide (PaCO₂) in arterial blood, oxygen saturation (SaO₂), and pH) were analyzed. For this purpose, arterial blood samples were obtained from control and experimental animals and analyzed within a few minutes using a test cartridge blood analysis system (Premier GEM 3500, Instrumentation Laboratory, Bedford, Mass., USA).

Isolation of Lung-Infiltrated Immune Cells

Lungs obtained from control and CS-exposed mice were washed with sterile phosphate-buffered saline (PBS) and placed in Petri dishes with DMEM supplemented with 8% FBS. The dissected lung tissues were incubated in a medium that contained collagenase type IV (0.5 mg/mL) and type IV bovine pancreatic DNAse (Roche Diagnostics; 1 mg/mL) at 37° C. for 45 minutes. The cells were filtered through a 100 μm nylon cell strainer into a clean 50 mL conical tube. Then, cells were pelleted by centrifuging for 10 min at 300 g at 10° C. Red blood cells were depleted with a lysis buffer (0.144 M NH4C1, 0.0169 M TRIS base, pH 7.4) at 37° C. in a 5% CO₂ atmosphere for 5 minutes.

Flow Cytometry Analysis and Intracellular Staining

Lung-infiltrated immune cells were screened for various cell surface and intracellular markers by flow cytometry. Since a combination of mechanical and enzymatic dissociations of lung tissue may result in cell damage and death, the MACS® Dead Cell Removal Kit (Miltenyi Biotec, Bergisch Gladbach, Germany) was used for magnetic cell separation of viable cells. Briefly, a single-cell suspension of lung-infiltrated cells was resuspended in 100 μL of the Dead Cell Removal MicroBeads (per 107 of cells), mixed, and incubated for 15 minutes at room temperature. Cells were applied on MS columns within 1×MACS® Binding Buffer. Effluent that passed through the column contained live cells. To reduce nonspecific binding of antibodies, viable lung-infiltrated cells were incubated with an anti-Fc block (anti-mouse CD16/CD32). For that purpose, the cell suspension was incubated with 1 μg of the BD Fc Block/106 cells in 100 μL of staining buffer (Dulbecco's PBS (DPBS) without Mg²⁺ or Ca²⁺, 1% heat-inactivated FCS, and 0.09% (w/v) sodium azide) for 15 minutes at 4° C. The cells were then washed and stained with fluorochrome-conjugated antibodies. Briefly, 1×10⁶ cells were incubated with anti-mouse CD45, F4/80, I-A, CD80, CD206, CD11c, NKp46, Gr-1, CD3, CD4, CD8, CXCR3, monoclonal antibodies conjugated with fluorescein isothiocyanate (FITC), phycoerythrin (PE), peridinin chlorophyll protein (PerCP), or allophycocyanin (APC) (all from BD Biosciences, San Jose, Calif., USA) in a staining buffer for 30 minutes in the dark at 4° C. Cells were washed twice in a staining buffer and pelleted by centrifugation. For intracellular cytokine staining, cells were stimulated with 50 ng/mL phorbol 12-myristate 13-acetate (PMA) and 500 ng/mL ionomycin for 5 hours and GolgiStop (BD Biosciences, San Jose, Calif., USA) was added. Cells were then incubated in a BD fixation/permeabilization solution (BD Cytofix/Cytoperm™ Fixation/Permeabilization Kit) for 20 minutes at 4° C. Afterwards, cells were washed two times in 1×BD Perm/Wash™ buffer (BD Cytofix/Cytoperm™ Fixation/Permeabilization Kit) and pelleted. Fixed/permeabilized cells were concomitantly resuspended in 50 μL of BD Perm/Wash™ buffer containing a predetermined optimal concentration of fluorochrome-conjugated antibodies specific for FoxP3, TNF-α, IL-12, IL-10, IL-1β, IFN-γ, and IL-17 by using appropriate anti-mouse monoclonal antibodies conjugated with FITC, PE, PerCP, and APC (BD Biosciences, San Jose, Calif., USA). Cells were incubated with fluorochrome-conjugated antibodies at 4° C. for 30 minutes in the dark. Afterwards, cells were washed 2 times with 1×BD Perm/Wash™ buffer and resuspended in a staining buffer prior to flow cytometric analysis. In experiments in which the phenotype and function of T cells were analyzed, CD3+ T lymphocytes were isolated from the population of viable lung-infiltrated cells by magnetic separation. For that purpose, the MACS Separator, the MACS Columns, and the CD3c MicroBead Kit, mouse (Miltenyi Biotec, Bergisch Gladbach, Germany) were used. Afterwards, CD3+ T cells were stained with fluorochrome-conjugated anti-mouse antibodies specific for CD4, CD8, CXCR3, FoxP3, TNF-α, IL-10, IFN-γ, and IL-17, following the procedure that was described above. Flow cytometric analysis was conducted on a BD Biosciences' FACSCalibur and analyzed by using the Flowing Software analysis program.

Determination of Cytokines

Commercial ELISA sets (R&D Systems, Minneapolis, Minn., USA) were used to determine the concentration of TNF-α, IL-12, IL-10, IL-1β, and IFN-γ in serum samples of control and experimental animals.

COPD Patients

Thirty COPD patients were recruited with the aim to receive an Exosomes d-MAPPS inhalation solution. Patients enrolled in this study were men (n=20) or postmenopausal women (n=10) aged between 50 and 75 years, having a postbronchodilator forced expiratory volume in 1 s (FEV1)≥30% and <80% predicted, a postbronchodilator FEV1/forced vital capacity (FVC)<70%, a smoking history of ≥10 packs per year, and lung hyperinflation defined as a functional residual capacity (FRC) greater than 120%. Subjects with past or current history of abnormal vital signs, abnormal laboratory findings, clinically relevant ECG abnormalities, or cardiovascular conditions prior to screening were excluded from the study. All subjects provided written informed consent prior to study participation.

Clinical Study Design

Patients received Exosomes d-MAPPS inhalation solution (0.5 mL/once per week for three weeks) containing a high concentration of immunosuppressive factors (soluble TNF receptors I and II (sTNFRI and sTNFRII), IL-1 receptor antagonist (IL-1Ra), and soluble receptor for advanced glycation end products (sRAGE)). Pulmonary function tests and clinical findings were recorded before, and 1 month after, such treatment. Spirometry was performed according to recommendations from the American Thoracic Society guidelines. Forced expiratory volume in 1 second (FEV1) and peak expiratory flow (PEF) rate were recorded. Chest computed tomography (CT), standard clinical COPD questionnaire (CCQ) scoring, and 6-minute walking distance (6MWD) test as a submaximal test of aerobic capacity/endurance were used to determine the effects of the treatment.

Statistical Analysis

The results obtained in the animal study were analyzed using the Student t-test. All data in animal studies were expressed as the mean±standard error of the mean (SEM). The Wilcoxon signed-rank test was applied to demonstrate differences in pulmonary function of COPD patients before and after Exosomes d-MAPPS treatment. Values of P<0.05 were considered statistically significant.

Results

Results from both the animal models and the human patients generally showed alleviation of chronic airway inflammation after treatment, as described in further detail below.

Chronic Airway Inflammation in Mice

Remarkably improved respiratory function, as evidenced by significantly elevated PaO₂ (P<0.0001), O₂ saturation (P<0.0001), and pH (P<0.0001) and decreased PaCO₂ (P<0.0001), was observed in CS-treated mice that received Exosomes d-MAPPS. Accordingly, depression-like behavior and loss of locomotor activity were not seen in CS+ Exosomes d-MAPPS-treated animals.

The alveolar wall was intact, and leucocyte accumulation was not seen, in the lung parenchyma of control animals. By contrast, partial alveolar wall destruction, widened alveolar septa and expanded alveolar space, capillary dilation, and congestion with massive infiltration of neutrophils, lymphocytes, and monocytes were observed in the lungs of CS-exposed mice. Importantly, preserved alveolar and blood vessel structures and a significantly lower number of lung-infiltrated leucocytes were noticed in the lungs of CS+ Exosomes d-MAPPS-treated animals, indicating that treatment managed to attenuate inflammation-related pathological changes in the lungs of CS-exposed mice.

In line with these findings, a significantly lower concentration of inflammatory cytokines that play an important pathogenic role in the development and progression of CS-induced airway inflammation (e.g., TNF-α, IL-1β, IL-12, and IFN-γ) was observed in serum samples of Exosomes d-MAPPS-treated CS-exposed mice compared to CS+ vehicle-treated animals (P<0.05 for TNF-α, IL-12, and IFN-γ; P<0.01 for IL-1β). Additionally, treatment resulted in the elevation of anti-inflammatory and immunosuppressive IL-10 (P<0.01), which is involved in lung repair and regeneration.

Inflammatory Macrophages, Neutrophils, and NK and NKT Cells in Inflamed Lungs

Treatment managed to significantly reduce the total number of lung-infiltrated macrophages in CS-exposed mice (P<0.001). Additionally, Exosomes d-MAPPS remarkably attenuated antigen-presenting capacities of alveolar macrophages, as evidenced by a significantly reduced number of CD80- and I-A-expressing F4/80+ cells in the lungs of CS+ Exosomes d-MAPPS-treated animals (P<0.001). Intracellular staining revealed that treatment significantly attenuated the production of inflammatory TNF-α (P<0.001) and IL-12 (P<0.01) in lung-infiltrated macrophages. Furthermore, a significantly higher number of alternatively activated, IL-10-producing and CD206-expressing M2 macrophages were noticed in the lungs of Exosomes d-MAPPS-treated CS-exposed mice (P<0.01), indicating that Exosomes d-MAPPS treatment suppressed inflammation and promoted the generation of an immunosuppressive phenotype in lung-infiltrated macrophages.

Additionally, treatment attenuated the capacity of NK and NKT cells and neutrophils to produce inflammatory cytokines in CS-injured lungs. A significantly lower number of IL-17A-producing NK and NKT cells (P<0.001 for NK and P<0.05 for NKT cells), IFN-γ-secreting NK and NKT cells (P<0.001), and TNF-α and IL-1β-producing neutrophils (P<0.001) were observed in the lungs of Exosomes d-MAPPS-treated CS-exposed mice.

Attenuated Activation of CD4+ and CD8+ T Lymphocytes

Exosomes d-MAPPS affected the migratory and antigen-presenting properties of DCs. A significantly lower number of F4/80-CD11c+I-A+ DCs were observed in the CS-injured lungs of treated animals (P<0.001). The total number of lung-infiltrated F4/80-CD11c+I-A+ DCs that expressed costimulatory molecule CD80 (P<0.01) was significantly lower in CS-treated mice that received Exosomes d-MAPPS. Furthermore, a decreased number of proinflammatory, IL-12-producing F4/80-CD11c+I-A+ DCs (P<0.001) and an increased presence of immunosuppressive and tolerogenic, IL-10-producing F4/80-CD11c+I-A+DCs (P<0.001) were observed in the lungs of CS+ Exosomes d-MAPPS-treated animals, indicating that the treatment attenuated the antigen-presenting and proinflammatory properties of airway DCs.

Exosomes d-MAPPS-induced modulation of DC function resulted in alleviated activation of inflammatory, IFN-γ- and IL-17-producing CD4+ and CD8+ T lymphocytes. A significantly lower number of CXCR3-expressing and IFN-γ-producing CD4+ Tₕ1 cells (P<0.01) and IL-17-producing CD4+ Tₕ17 cells (P<0.01) were observed in the lungs of treated CS-exposed mice. Similarly, Exosomes d-MAPPS treatment attenuated the influx of CXCR-expressing, IFN-γ-producing (P<0.001), and IL-17-producing CD8+CTLs (P<0.01) and reduced the total number of alveolotoxic, TNF-α-producing CD8+CTLs (P<0.001) in CS-injured lungs. Importantly, treatment significantly increased the total number of lung-infiltrated anti-inflammatory, IL-10-producing CD4+FoxP3+ regulatory T cells (Tregs) (P<0.05), enabling the generation of an immunosuppressive microenvironment in the inflamed lungs.

Improved Pulmonary Status of COPD Patients

Exosomes d-MAPPS contained a high concentration of soluble immunosuppressive mediators (e.g., sTNFRI, sTNFRII, IL-1Ra, and sRAGE). Clinical parameters and CT findings indicated the beneficial effects of Exosomes d-MAPPS in the alleviation of chronic lung inflammation. All of the 30 treated patients showed a marked improvement in pulmonary status, as evidenced by an increase in percentage change relative to the initial value of FEV1 (%ΔFEV1), significantly higher PEF, decreased CCQ total score, and increased 6-minute walking distance (6MWD). Additionally, quality of life was significantly improved after treatment and all treated patients managed to perform daily activities without hindrance. Clinical findings were confirmed by CT. Inflammation-induced destruction of alveoli and air trapping caused hyperinflation of the lungs with flattening of the diaphragm in COPD patients. Exosomes d-MAPPS significantly alleviated emphysematous changes in the lungs of COPD patients. Lungs were less hyperexpanded, diaphragms were less flattened, and centrilobular and paraseptal emphysema were significantly reduced one month after Exosomes d-MAPPS administration, indicating the beneficial effects of treatment in the attenuation of emphysema in COPD patients. Importantly, Exosomes d-MAPPS was well tolerated. None of the 30 treated COPD patients reported any side effects related to Exosomes d-MAPPS administration.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

What is claimed is:

1. A method for treatment of cancers and/or tumors in a subject, the method comprising:
administering to the subject an effective amount of a pharmaceutical composition and/or formulation comprising one or more types of mesenchymal stem cells (MSCs),
wherein the one or more types of MSCs alter one or more responses of one or more immune cells in the subject.

2. The method of claim 1, wherein the one or more immune cells are selected from the group consisting of: dendritic cells, macrophages, T cells, natural killer (NK) cells, and combinations thereof.

3. The method of claim 1, wherein the altering one or more responses of one or more immune cells in the subject comprises enhancing and/or inducing one or more endogenous immune cells in one or more tumors present in the subject.

4. The method of claim 3, wherein the enhancing and/or inducing comprises at least one of:
enhancing cytotoxicity of one or more CD8+ cytotoxic T lymphocytes (CTLs),
enhancing cytotoxicity of one or more NK cells,
increasing production of one or more cytokines in CD4+ Tₕ1 lymphocytes, and
increasing production of one or more cytokines in CD4+ Tₕ17 lymphocytes.

5. The method of claim 1, wherein the one or more types of MSCs are infected with one or more viruses selected from the group consisting of: oncolytic adenoviruses, oncolytic herpes simplex virus (HSV), oncolytic measles virus, and combinations thereof.

6. The method of claim 1, further comprising:
administering to the subject one or more doses of radiation, wherein the radiation is ultraviolet (UV) radiation and/or X-ray irradiation.

7. The method of claim 1, further comprising:
administering to the subject one or more additional agents in combination with the pharmaceutical composition and/or formulation, wherein the one or more additional agents are selected from the group consisting of: gemcitabine (GCB), paclitaxel (PTX), doxorubicin (DOX), 5-fluorocytosine (5-FC), ganciclovir (GCV), an adjuvant, an antigen, an excipient, a vaccine, an allergen, an antibiotic, a gene therapy vector, a kinase inhibitor, a co-stimulatory molecule, a Toll-like receptor (TLR) agonist, a TLR antagonist, a therapeutic agent, a prophylactic agent, a diagnostic agent, an antimicrobial agent, an analgesic, a local anesthetic, an anti-inflammatory agent, an anti-oxidant agent, an immunosuppressant agent, an anti-allergenic agent, an enzyme cofactor, an essential nutrient, a growth factor, and combinations thereof.

8. The method of claim 1, wherein the administering to the subject an effective amount of a pharmaceutical composition and/or formulation comprising one or more types of MSCs further comprises:
administering, with the pharmaceutical composition and/or formulation, a pharmaceutically acceptable carrier for injection.

9. The method of claim 8, wherein the administering to the subject an effective amount of a pharmaceutical composition and/or formulation comprising one or more types of MSCs prevents cancer in the subject and/or decreases incidence of cancer in the subject.

10. The method of claim 1, wherein the administering to the subject an effective amount of a pharmaceutical composition and/or formulation comprising one or more types of MSCs reduces tumor weight and/or tumor volume in the subject.

11. The method of claim 1, wherein the administering to the subject an effective amount of a pharmaceutical composition and/or formulation comprising one or more types of MSCs further comprises:
administering the pharmaceutical composition and/or formulation systemically and/or at tumor locations in the subject.

12. The method of claim 1, wherein the administering to the subject an effective amount of a pharmaceutical composition and/or formulation comprising one or more types of MSCs results in at least one of:
an increased number of cytotoxic NK cells expressing at least one of: interferon gamma (IFN-γ), Fas ligand (FASL), and granzyme B,
an increased number of CD4+ helper T cells, and
an increased number of CD8+ cytotoxic T lymphocytes (CTLs).

13. The method of claim 1, wherein the administering to the subject an effective amount of a pharmaceutical composition and/or formulation comprising one or more types of MSCs results in at least one MSC in the one or more types of MSCs having an anti-tumorigenic MSC1 phenotype, wherein the at least one MSC having the anti-tumorigenic MSC1 phenotype secretes tumor necrosis factor-alpha (TNF-α) and/or interferon-gamma (IFN-γ).

14. A method of treatment of cancers and/or tumors in a subject, the method comprising:
administering to the subject an effective amount of a pharmaceutical composition and/or formulation comprising one or more types of mesenchymal stem cell (MSC)-derived exosomes,
wherein the one or more types of MSC-derived exosomes alter one or more responses of one or more immune cells in the subject, and
wherein the one or more types of MSC-derived exosomes are derived ex vivo from one or more types of MSCs.

15. The method of claim 14, wherein the one or more types of MSC-derived exosomes secretes comprises one or more anti-tumorigenic microRNAs selected from the group consisting of: miRNA-16-5p, miRNA-3940-5p, miRNA-4461, miRNA-15a, miRNA-100, and combinations thereof.

16. The method of claim 14, wherein the administering to the subject an effective amount of a pharmaceutical composition and/or formulation comprising one or more types of MSC-derived exosomes results in an increased concentration of at least one of: IL-1Ra, CCL21, CXCR6, and CXCL14.

17. The method of claim 14, wherein the administering to the subject an effective amount of a pharmaceutical composition and/or formulation comprising one or more types of MSC-derived exosomes results in downregulation of vascular endothelial growth factors (VEGF) production.

18. A pharmaceutical composition comprising:
one or more types of mesenchymal stem cells (MSCs), and
one or more pharmaceutically acceptable excipients.

19. The pharmaceutical composition of claim 18, further comprising:
one or more agents selected from the group consisting of: an adjuvant, an antigen, an excipient, a vaccine, an allergen, an antibiotic, a gene therapy vector, a kinase inhibitor, a co-stimulatory molecule, a Toll-like receptor (TLR) agonist, a TLR antagonist, a therapeutic agent, a prophylactic agent, a diagnostic agent, an antimicrobial agent, an analgesic, a local anesthetic, an anti-inflammatory agent, an anti-oxidant agent, an immunosuppressant agent, an anti-allergenic agent, an enzyme cofactor, an essential nutrient, a growth factor, and combinations thereof.

20. The pharmaceutical composition of claim 18, further comprising:
one or more exosomes generated ex vivo from one or more types of MSCs.
